SEQUENCE LISTING (155 sequences)

SEQ ID NO: 1 - protein, 5 aa, Mus musculus
SHWLH

SEQ ID NO: 2 - protein, 17 aa, Mus musculus
VLDPSDFYSNYNQNFKG

SEQ ID NO: 3 - protein, 11 aa, mus musculus
GLLSGDYAMDY

SEQ ID NO: 4 - protein, 16 aa, mus musculus
RSSQSIVHSNGNTYLE

SEQ ID NO: 5 - protein, 7 aa, mus musculus
KVSNRFS

SEQ ID NO: 6 - protein, 9 aa, mus musculus
FQGSYVPWT

SEQ ID NO: 7 - protein, 120 aa, mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGAGTSVTVSS

SEQ ID NO: 8 - protein, 112 aa, mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIK

SEQ ID NO: 9 - protein, 463 aa, mus musculus
MGWSCIIVLLVSTATGVHSQVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 238 aa, mus musculus
MKLPVRLLVLMFWIPASSSDVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 11 - protein, 444 aa, mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 219 aa, mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 13 - protein, 10 aa, mus musculus
QVQLQQPGAE

SEQ ID NO: 14 - protein, 10 aa, mus musculus
DVLMTQIPLS

SEQ ID NO: 15 - protein, 5 aa, Artificial Sequence
  Description of Artificial Sequence Synthetic peptide
DYAMS

SEQ ID NO: 16 - protein, 17 aa, Artificial Sequence
  Description of Artificial Sequence Synthetic peptide
TISDGGTYTYYPDSVKG

SEQ ID NO: 17 - protein, 10 aa, Artificial Sequence
  Description of Artificial Sequence Synthetic peptide
EWGDYDGFDY

SEQ ID NO: 18 - protein, 11 aa, Artificial Sequence
  Description of Artificial Sequence Synthetic peptide
RASQEISGYLS

SEQ ID NO: 19 - protein, 7 aa, Artificial Sequence
  Description of Artificial Sequence Synthetic peptide
AASTLDS

SEQ ID NO: 20 - protein, 9 aa, Artificial Sequence
  Description of Artificial Sequence Synthetic peptide
LQYDSYPYT

SEQ ID NO: 21 - protein, 119 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 22 - protein, 107 aa, Artificial Sequence
  Description of Artificial Sequence Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 23 - protein, 471 aa, Artificial Sequence
  Description of Artificial Sequence Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 236 aa, Artificial Sequence
  Description of Artificial Sequence Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 5 aa, Artificial Sequence
  Description of Artificial Sequence Synthetic peptide
SHWLH

SEQ ID NO: 26 - protein, 17 aa, Artificial Sequence
  Description of Artificial Sequence Synthetic peptide
VLDPSDFYSNYNQNFKG

SEQ ID NO: 27 - protein, 11 aa, Artificial Sequence
  Description of Artificial Sequence Synthetic peptide
GLLSGDYAMDY

SEQ ID NO: 28 - protein, 16 aa, Artificial Sequence
  Description of Artificial Sequence Synthetic peptide
RSSQSIVHSNGNTYLE

SEQ ID NO: 29 - protein, 7 aa, Artificial Sequence
  Description of Artificial Sequence Synthetic peptide
KVSNRFS

SEQ ID NO: 30 - protein, 9 aa, Artificial Sequence
  Description of Artificial Sequence Synthetic peptide
FQGSYVPWT

SEQ ID NO: 31 - protein, 120 aa, Artificial Sequence
  Description of Artificial Sequence Synthetic polypeptide
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSS

SEQ ID NO: 32 - protein, 112 aa, Artificial Sequence; identical to SEQ ID NO: 8
  Description of Artificial Sequence Synthetic polypeptide

SEQ ID NO: 33 - protein, 463 aa, Artificial Sequence; identical to SEQ ID NO: 9
  Description of Artificial Sequence Synthetic polypeptide

SEQ ID NO: 34 - protein, 238 aa, Artificial Sequence; identical to SEQ ID NO: 10
  Description of Artificial Sequence Synthetic polypeptide

SEQ ID NO: 35 - protein, 7 aa, Artificial Sequence
  Description of Artificial Sequence Synthetic peptide
TFGLSVG

SEQ ID NO: 36 - protein, 16 aa, Artificial Sequence
  Description of Artificial Sequence Synthetic peptide
HIWWDDDKYYNPALKS

SEQ ID NO: 37 - protein, 10 aa, Artificial Sequence
  Description of Artificial Sequence Synthetic peptide
IGADALPFDY

SEQ ID NO: 38 - protein, 16 aa, Artificial Sequence
  Description of Artificial Sequence Synthetic peptide
RSSKSLLHSNGNTYLY

SEQ ID NO: 39 - protein, 7 aa, Artificial Sequence
  Description of Artificial Sequence Synthetic peptide
RMSNLAS

SEQ ID NO: 40 - protein, 9 aa, Artificial Sequence
  Description of Artificial Sequence Synthetic peptide
MQHLEYPFT

SEQ ID NO: 41 - protein, 120 aa, Artificial Sequence
  Description of Artificial Sequence Synthetic polypeptide
QVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSS

SEQ ID NO: 42 - protein, 112 aa, Artificial Sequence
  Description of Artificial Sequence Synthetic polypeptide
DIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 43 - protein, 475 aa, Artificial Sequence
  Description of Artificial Sequence Synthetic polypeptide
MGRLTSSFLLLIVPAYVLSQVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 44 - protein, 239 aa, Artificial Sequence
  Description of Artificial Sequence Synthetic polypeptide
MRCLAEFLGLLVLWIPGAIGDIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 45 - protein, 5 aa, Artificial Sequence
  Description of Artificial Sequence Synthetic peptide
DHIIH

SEQ ID NO: 46 - protein, 17 aa, Artificial Sequence
  Description of Artificial Sequence Synthetic peptide
YIYPRDGYIKYNEKFKG

SEQ ID NO: 47 - protein, 8 aa, Artificial Sequence
  Description of Artificial Sequence Synthetic peptide
GYYYAMDY

SEQ ID NO: 48 - protein, 16 aa, Artificial Sequence
  Description of Artificial Sequence Synthetic peptide
RSSQSIVHSIGNTYLE

SEQ ID NO: 49 - protein, 9 aa, Artificial Sequence
  Description of Artificial Sequence Synthetic peptide
FQGSHVPFT

SEQ ID NO: 50 - protein, 117 aa, Artificial Sequence
  Description of Artificial Sequence Synthetic polypeptide
QVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSS

SEQ ID NO: 51 - protein, 112 aa, Artificial Sequence
  Description of Artificial Sequence Synthetic polypeptide
DVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIK

SEQ ID NO: 52 - protein, 460 aa, Artificial Sequence
  Description of Artificial Sequence Synthetic polypeptide
MEWSWVSLFFLSVTTGVHSQVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 238 aa, Artificial Sequence
  Description of Artificial Sequence Synthetic polypeptide
MKLPVRLLVLMFWIPASRSDVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 54 - protein, 5 aa, Artificial Sequence
  Description of Artificial Sequence Synthetic peptide
SYWMH

SEQ ID NO: 55 - protein, 17 aa, Artificial Sequence
  Description of Artificial Sequence Synthetic peptide
MIDPSDVYTNYNPKFKG

SEQ ID NO: 56 - protein, 6 aa, Artificial Sequence
  Description of Artificial Sequence Synthetic peptide
NYSGDY

SEQ ID NO: 57 - protein, 115 aa, Artificial Sequence
  Description of Artificial Sequence Synthetic polypeptide
QVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSS

SEQ ID NO: 58 - protein, 112 aa, Artificial Sequence
  Description of Artificial Sequence Synthetic polypeptide
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 59 - protein, 458 aa, Artificial Sequence
  Description of Artificial Sequence Synthetic polypeptide
MGWSCIIVLLVSTATCVHSQVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 60 - protein, 238 aa, Artificial Sequence
  Description of Artificial Sequence Synthetic polypeptide
MKLPVRLLVLMFWIPASSSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 61 - protein, 5 aa, Artificial Sequence
  Description of Artificial Sequence Synthetic peptide
TYGMS

SEQ ID NO: 62 - protein, 17 aa, Artificial Sequence
  Description of Artificial Sequence Synthetic peptide
WINTYSGVPTYADDFKG

SEQ ID NO: 63 - protein, 12 aa, Artificial Sequence
  Description of Artificial Sequence Synthetic peptide
GRDGYQVAWFAY

SEQ ID NO: 64 - protein, 11 aa, Artificial Sequence
  Description of Artificial Sequence Synthetic peptide
ITSTDIDDDMN

SEQ ID NO: 65 - protein, 7 aa, Artificial Sequence
  Description of Artificial Sequence Synthetic peptide
EGNTLRP

SEQ ID NO: 66 - protein, 9 aa, Artificial Sequence
  Description of Artificial Sequence Synthetic peptide
LQSDNLPYT

SEQ ID NO: 67 - protein, 121 aa, Artificial Sequence
  Description of Artificial Sequence Synthetic polypeptide
QIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSA

SEQ ID NO: 68 - protein, 107 aa, Artificial Sequence
  Description of Artificial Sequence Synthetic polypeptide
ETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIK

SEQ ID NO: 69 - protein, 464 aa, Artificial Sequence
  Description of Artificial Sequence Synthetic polypeptide
MGWLWNLLFLMAAAQSAQAQIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 234 aa, Artificial Sequence
  Description of Artificial Sequence Synthetic polypeptide
MFSLALLLSLLLLCVSDSRAETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 71 - protein, 5 aa, Artificial Sequence
  Description of Artificial Sequence Synthetic peptide
NYWMH

SEQ ID NO: 72 - protein, 17 aa, Artificial Sequence
  Description of Artificial Sequence Synthetic peptide
MIDPSDSYTNYNPKFKG

SEQ ID NO: 73 - protein, 115 aa, Artificial Sequence
  Description of Artificial Sequence Synthetic polypeptide
QVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSS

SEQ ID NO: 74 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 75 - protein, 458 aa, Artificial Sequence
  Description of Artificial Sequence Synthetic polypeptide
MGWSCIIVLLVSTATGVHSQVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 76 - protein, 238 aa, Artificial Sequence
  Description of Artificial Sequence Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 77 - protein, 449 aa, Artificial Sequence
  Antibody heavy chain
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, Artificial Sequence
  Antibody light chain
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 119 aa, Artificial Sequence
  Description of Artificial Sequence Synthetic polypeptide
EVQLVESGGGLVQPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 80 - protein, 119 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 81 - protein, 119 aa, Artificial Sequence; identical to SEQ ID NO: 21
  Description of Artificial Sequence Synthetic polypeptide

SEQ ID NO: 82 - protein, 119 aa, Artificial Sequence
  Description of Artificial Sequence Synthetic polypeptide
EVQLVESGGGLVKPGGSLRLSCAASGFTFSDYAMSWVRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 83 - protein, 119 aa, Artificial Sequence
  Description of Artificial Sequence Synthetic polypeptide
EVQLLESGGGLVQPGGSLRLSCAASGFTFSDYAMSWVRQAPGKGLEWVSTISDGGTYTYYPDNVKGRFTISRDNSKNTLYLQMNSLRAEDTAVYYCAREWGDYDGFDYWGQGTLVTVSS

SEQ ID NO: 84 - protein, 119 aa, Artificial Sequence
  Description of Artificial Sequence Synthetic polypeptide
QVQLVESGGGVVQPGRSLRLSCAASGFTFSDYAMSWVRQAPGKGLEWVATISDGGTYTYYPDNVKGRFTISRDNSKNTLYLQMSSLRAEDTAVYYCAREWGDYDGFDYWGQGTLVTVSS

SEQ ID NO: 85 - protein, 119 aa, Artificial Sequence
  Description of Artificial Sequence Synthetic polypeptide
EVQLVESGGGLVKPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 86 - protein, 107 aa, Artificial Sequence
  Description of Artificial Sequence Synthetic polypeptide
DIQLTQSPSFLSASVGDRVTITCRASQEISGYLSWYQQKPGKAPKLLIYAASTLDSGVPSRFSGSGSGTEFTLTISSLQPEDFATYYCLQYDSYPYTFGQGTKLEIK

SEQ ID NO: 87 - protein, 107 aa, Artificial Sequence
  Description of Artificial Sequence Synthetic polypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIK

SEQ ID NO: 88 - protein, 107 aa, Artificial Sequence; identical to SEQ ID NO: 22
  Description of Artificial Sequence Synthetic polypeptide

SEQ ID NO: 89 - protein, 107 aa, Artificial Sequence
  Description of Artificial Sequence Synthetic polypeptide
DIQMTQSPSSLSASVGDRVTITCRASQEISGYLSWYQQKPGKAPKLLIYAASTLDSGVPSRFSGSGSGTDFTFTISSLQPEDIATYYCLQYDSYPYTFGQGTKLEIK

SEQ ID NO: 90 - protein, 107 aa, Artificial Sequence
  Description of Artificial Sequence Synthetic polypeptide
DIQMTQSPSSLSASVGDRVTITCRASQEISGYLSWYQQKPGKAPKLLIYAASTLDSGVPSRFSGSGSGTDFTLTISSLQPEDFATYYCLQYDSYPYTFGQGTKLEIK

SEQ ID NO: 91 - protein, 107 aa, Artificial Sequence
  Description of Artificial Sequence Synthetic polypeptide
DIQMTQSPSSLSASVGDRVTITCRASQEISGYLSWLQQKPGGAIKRLIYAASTLDSGVPSRFSGSGSGSDYTLTISSLQPEDFATYYCLQYDSYPYTFGQGTKLEIK

SEQ ID NO: 92 - protein, 468 aa, Artificial Sequence
  Description of Artificial Sequence Synthetic polypeptide
MNFGLSLMFLVLVLKGVQCEVQLVESGGGLVKPGGSLKLSCAASGFTFSDYAMSWVRQTPEKRLEWVATISDGGTYTYYPDNVKGRFTISRDNAKNNLYLQMSHLKSEDTAMYYCAREWGDYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 236 aa, Artificial Sequence
  Description of Artificial Sequence Synthetic polypeptide
MDMRVPAHVFGFLLLWFPGTRCDIQMTQSPSSLSASLGERVSLTCRASQEISGYLSWLQQKPDGTIKRLIYAASTLDSGVPKRFSGSRSGSDYSLTIGSLESEDLADYYCLQYDSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 471 aa, Artificial Sequence; identical to SEQ ID NO: 23
  Description of Artificial Sequence Synthetic polypeptide

SEQ ID NO: 95 - protein, 467 aa, Artificial Sequence
  Description of Artificial Sequence Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 236 aa, Artificial Sequence
  Description of Artificial Sequence Synthetic polypeptide
MDMRVPAQLLGLLLLWLRGARCDIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 236 aa, Artificial Sequence; identical to SEQ ID NO: 24
  Description of Artificial Sequence Synthetic polypeptide

SEQ ID NO: 98 - protein, 449 aa, Artificial Sequence
  Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 445 aa, Artificial Sequence
  Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 214 aa, Artificial Sequence
  Antibody light chain no leader sequence
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 214 aa, Artificial Sequence; identical to SEQ ID NO: 78
  Antibody light chain no leader sequence

SEQ ID NO: 102 - protein, 450 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, Artificial Sequence
  Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 448 aa, Artificial Sequence
  Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 214 aa, Artificial Sequence
  Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 447 aa, Artificial Sequence
  Antibody heavy chain
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 220 aa, Artificial Sequence
  Antibody light chain
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 445 aa, Artificial Sequence
  Antibody heavy chain
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 109 - protein, 217 aa, Artificial Sequence
  Antibody light chain
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 110 - protein, 449 aa, Artificial Sequence
  Antibody heavy chain
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 220 aa, Artificial Sequence
  Antibody light chain
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 446 aa, Artificial Sequence
  Antibody heavy chain
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 214 aa, Artificial Sequence
  Antibody light chain
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 25 bp, Artificial Sequence
  Morpholino
cgtcacaggcaggacccactgccca

SEQ ID NO: 115 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 116 - protein, 345 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 345 aa, Artificial Sequence; identical to SEQ ID NO: 116
  Fusion protein

SEQ ID NO: 118 - protein, 347 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 471 aa, Artificial Sequence; identical to SEQ ID NO: 23
  Description of Artificial Sequence Synthetic polypeptide

SEQ ID NO: 120 - protein, 236 aa, Artificial Sequence; identical to SEQ ID NO: 24
  Description of Artificial Sequence Synthetic polypeptide

SEQ ID NO: 121 - protein, 463 aa, Artificial Sequence; identical to SEQ ID NO: 9
  Description of Artificial Sequence Synthetic polypeptide

SEQ ID NO: 122 - protein, 238 aa, Artificial Sequence; identical to SEQ ID NO: 10
  Description of Artificial Sequence Synthetic polypeptide

SEQ ID NO: 123 - protein, 475 aa, Artificial Sequence; identical to SEQ ID NO: 43
  Description of Artificial Sequence Synthetic polypeptide

SEQ ID NO: 124 - protein, 239 aa, Artificial Sequence; identical to SEQ ID NO: 44
  Description of Artificial Sequence Synthetic polypeptide

SEQ ID NO: 125 - protein, 460 aa, Artificial Sequence
  Description of Artificial Sequence Synthetic polypeptide
MEWSWVSLFFLSVTTGVHSQVQLQQSDAELVKPGASVKISCKVSGYTFTDHIIHWMKQRPEQGLEWIGYIYPRDGYIKYNYFCARGYYYAMDYWGQEKFKGKATLTADKSSSTAYMQVNSLTSEDSAVGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 126 - protein, 238 aa, Artificial Sequence; identical to SEQ ID NO: 53
  Description of Artificial Sequence Synthetic polypeptide

SEQ ID NO: 127 - protein, 458 aa, Artificial Sequence; identical to SEQ ID NO: 59
  Description of Artificial Sequence Synthetic polypeptide

SEQ ID NO: 128 - protein, 238 aa, Artificial Sequence; identical to SEQ ID NO: 60
  Description of Artificial Sequence Synthetic polypeptide

SEQ ID NO: 129 - protein, 464 aa, Artificial Sequence; identical to SEQ ID NO: 69
  Description of Artificial Sequence Synthetic polypeptide

SEQ ID NO: 130 - protein, 234 aa, Artificial Sequence; identical to SEQ ID NO: 70
  Description of Artificial Sequence Synthetic polypeptide

SEQ ID NO: 131 - protein, 458 aa, Artificial Sequence; identical to SEQ ID NO: 75
  Description of Artificial Sequence Synthetic polypeptide

SEQ ID NO: 132 - protein, 238 aa, Artificial Sequence; identical to SEQ ID NO: 76
  Description of Artificial Sequence Synthetic polypeptide

SEQ ID NO: 133 - protein, 449 aa, Artificial Sequence; identical to SEQ ID NO: 77
  Antibody heavy chain

SEQ ID NO: 134 - protein, 214 aa, Artificial Sequence; identical to SEQ ID NO: 78
  Antibody light chain

SEQ ID NO: 135 - protein, 451 aa, Artificial Sequence
  mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 121 aa, Artificial Sequence
  mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 137 - protein, 5 aa, Artificial Sequence
  mAb
NYGMN

SEQ ID NO: 138 - protein, 17 aa, Artificial Sequence
  mAb
WINTYTGEPTYTDDFKG

SEQ ID NO: 139 - protein, 12 aa, Artificial Sequence
  mAb
GGFGSSYWYFDV

SEQ ID NO: 140 - protein, 214 aa, Artificial Sequence
  mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 110 aa, Artificial Sequence
  mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTV

SEQ ID NO: 142 - protein, 11 aa, Artificial Sequence
  mAb
KASQDVSIAVA

SEQ ID NO: 143 - protein, 7 aa, Artificial Sequence
  mAb
SASYRYT

SEQ ID NO: 144 - protein, 9 aa, Artificial Sequence
  mAb
QQHYITPLT

SEQ ID NO: 145 - protein, 451 aa, Artificial Sequence
  mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 121 aa, Artificial Sequence
  mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 147 - protein, 5 aa, Artificial Sequence
  mAb
TAGMQ

SEQ ID NO: 148 - protein, 17 aa, Artificial Sequence
  mAb
WINTHSGVPKYAEDFKG

SEQ ID NO: 149 - protein, 12 aa, Artificial Sequence
  mAb
SGFGSSYWYFDV

SEQ ID NO: 150 - protein, 214 aa, Artificial Sequence
  mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 110 aa, Artificial Sequence
  mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTV

SEQ ID NO: 152 - protein, 11 aa, Artificial Sequence
  mAb
KASQDVSTAVA

SEQ ID NO: 153 - protein, 7 aa, Artificial Sequence
  mAb
SASYRYT

SEQ ID NO: 154 - protein, 9 aa, Artificial Sequence
  mAb
QQHYITPLT

SEQ ID NO: 155 - protein, 382 aa, Artificial Sequence
  fusion protein
EPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

(19) United States
(12) Patent Application Publication
(10) Pub. No.: US 2022/0251239 A1
(43) Pub. Date: Aug. 11, 2022

(54) COMBINATION RADIOIMMUNOTHERAPY AND CD47 BLOCKADE IN THE TREATMENT OF CANCER

(71) Applicant: ACTINIUM PHARMACEUTICALS, INC., New York, NY (US)

(72) Inventors: Dale L Ludwig, Rockaway, NJ (US); Sandesh Seth, New York, NY (US); Paul Diamond, Fort Lee, NJ (US)

(21) Appl. No.: 17/725,544

(22) Filed: Apr. 20, 2022

Related U.S. Application Data
(63) Continuation-in-part of application No. 17/702,648, filed on Mar. 23, 2022.
Application No. 17/702,648 is a continuation-in-part of application No. PCT/US2021/056259, filed on Oct. 21, 2021.
(60) Provisional application No. 63/250,725, filed on Sep. 30, 2021.
(60) Provisional application No. 63/226,699, filed on Jul. 28, 2021.
(60) Provisional application No. 63/104,386, filed on Oct. 22, 2020.

(51) Int. Cl. C07K 16/32; A61K 51/10; C07K 16/28; A61P 35/02; A61P 35/00

(52) U.S. Cl. CPC C07K 16/32; A61K 51/1045; C07K 16/2803; A61P 35/02; A61K 2039/507; A61K 51/1072; A61K 51/1069; A61K 51/1057; A61P 35/00

(57) ABSTRACT

Provided are compositions and methods for treating cancers and precancerous proliferative disorders in a mammalian subject that involve the combination use of a radiotherapeutic agent, such as a radiolabeled CD33, DR5, 5T4, HER2, HER3, or TROP2 targeting agent, and a CD47 checkpoint inhibitor, such as a SIRPα-IgG Fc fusion protein or a monoclonal antibody against CD47 or SIRPα.

### CROSS-REFERENCE TO RELATED APPLICATIONS

This application is a continuation-in-part of U.S. application Ser. No. 17/702,648 filed Mar. 23, 2022 which is a continuation-in-part of International application no. PCT/US2021/056259 filed Oct. 21, 2021, which claims priority to U.S. provisional application Ser. No. 63,250,725 filed Sep. 30, 2021, 63/226,699 filed Jul. 28, 2021, and 63/104,386 filed Oct. 22, 2020, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 20, 2022, is named ATNM-001PCT-CIP-CIP_SL_ST25.txt and is 262,646 bytes in size.

### FIELD OF THE INVENTION

The presently claimed invention relates to the field of radiotherapeutics.

### BACKGROUND

CD47 is an integrin-associated transmembrane protein that is ubiquitously expressed on the surface of both normal and malignant tissues. The binding of CD47 to its cognate receptor partner, signal receptor protein-alpha (SIRPα), found on phagocytes such as macrophages and dendritic cells results in an inhibition of phagocytosis. CD47 therefore provides a “don't eat me” signal to the phagocytes.

CD47 is expressed on virtually all normal cells, including red blood cells even though they do not express integrins. This pathway has evolved as a natural process by which the immune system can effectively and selectively clear aged, dead, or dying cells, but leave normal cells alone. To this end, CD47 is frequently overexpressed on the surface of many types of tumors as a means of immune evasion to avoid engulfment and clearance of tumor cells. Suppression of CD47 engagement of SIRPα by therapeutic blocking antibodies leads to the enablement of phagocytosis.

Suppression of the don't eat me signal by CD47, however, is insufficient to trigger macrophage phagocytosis. Under normal physiologic conditions, cellular homeostasis is partly regulated by balancing pro- and anti-phagocytic signals. For target cells to be phagocytosed upon CD47 blockade, the cells must also display a potent pro-phagocytic signal, the main “eat me” signals being elicited by surface expressed calreticulin and phosphatidylserine. This is a process for engulfment and removal of dead or dying cells. Significantly, blockade of the CD47-SIRPα interaction to facilitate tumor cell engulfment is an emerging therapeutic strategy in the treatment of many types of cancer. However, clinical responses to single agent therapeutics such as treatment with an anti-CD47 blocking antibody therapy have been modest.

What is needed and provided by the present invention are new therapeutic approaches for the treatment of proliferative disorders, such as cancers and precancerous proliferative disorders, that include the administration of one or more radiolabeled cancer targeting agents and one or more CD47 blocking agents.

### SUMMARY OF THE INVENTION

The presently disclosed invention is based on the discovery that administration of a combination including at least one radiotherapeutic, such as a radiolabeled cancer-associated antigen-targeting agent, and a CD47 blockade tips the balance of the pro- and anti-phagocytic signals toward phagocytosis for cancer cells. More specifically, the combination of radioimmunotherapies such as a radiolabeled targeting agent directed against a cancer-associated antigen such as CD33, DR5, 5T4, HER2, HER3, TROP2 or any of those disclosed herein and a CD47 blocking agent, such as a blocking monoclonal antibody against CD47 or SIRPα, may enhance clinical outcomes for cancer patients, including those with solid tumor cancers or hematological malignancies.

Accordingly, the present invention provides compositions and methods for treating a subject having a proliferative disorder such as cancer or a precancerous proliferative order. The compositions generally include a radiotherapeutic agent and a CD47 blockade. Exemplary radiotherapeutic agents include a radiolabeled targeting agent directed against CD33, DR5, 5T4, HER2, HER3, or TROP2 such as a radiolabeled antibody, peptide, or small molecule that binds specifically to CD33, DR5, 5T4, HER2, HER3, or TROP2. Exemplary CD33 targeting agents include any one or more of the monoclonal anti-CD33 antibodies lintuzumab, gemtuzumab, or vadastuximab, such as ²²⁵Ac-lintuzumab. Exemplary DR5 targeting agents include any one or more of the monoclonal anti-DR5 antibodies mapatumumab, conatumumab, lexatumumab, tigatuzumab, drozitumab, and LBY-135. Exemplary 5T4 targeting agents include any one or more of the monoclonal anti-5T4 antibodies MED10641, ALG.APV-527, Tb535, H6-DM5, and ZV0508. Exemplary HER3 targeting agents may bind to an epitope of HER3 recognized by HER3 recognized by patritumab, seribantumab, lumretuzumab, elgemtumab, GSK2849330, or AV-203. Exemplary TROP2 targeting agents include the monoclonal antibodies Sacituzumab and Datopotamab, and antibodies recognizing the same epitope of TROP2 recognized by either of said antibodies. Exemplary CD47 blockades include agents capable of blocking CD47 binding to SIRPα, such as magrolimab, lemzoparlimab, AO-176, ALX148, TTI-621, or TTI-622, as well as nucleic acid based modulators such as MBT-001 and small molecule modulators such as RRx-001.

According to certain aspects, the radiotherapeutic includes an actinium labeled monoclonal antibody against CD33, DR5, 5T4, HER2, HER3, or TROP2 administered in a radiation dose of 0.1 to 10 μCi/kg body weight of the subject and a protein dose of less than 10 mg/kg body weight of the subject. The CD47 blocking agent may, for example, include a monoclonal antibody that prevents CD47 binding to SIRPα. The CD47 blockade may, for example, include magrolimab, lemzoparlimab, AO-176, AK117, IMC-002, IBI-188, IBI-322, BI 766063, ZL-1201, AXL148, RRx-001, Azelnidipine, ES004, SRF231, SHR-1603, TJC4, TTI-621, or TTI-622. Exemplary effective doses for the CD47 blockade include 0.05 to 50 mg/kg, such as 0.05 to 5 mg/kg patient weight, or the doses approved for drug use or clinical trials of the agents. Exemplary doses of RRx-001 include 5-80 mg/m² or any subrange between integer values therein, such as 10-50 mg/m², 10-30 mg/m², or 10-20 mg/m², or any whole integer numerical value in said range±5%.

According to certain aspects, the cancer may be a solid tumor or a hematological cancer such as a myeloid malignancy. Exemplary myeloid malignancies include multiple myeloma, acute myelogenous leukemia, chronic myelogenous leukemia, myelodysplastic syndrome, or myeloproliferative neoplasm. According to certain aspects, the cancer may be associated with CD33 positive cells, such as myeloblast cells or malignant plasmacytes.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 is a graph showing the comparative effects on tumor growth of vehicle (control), magrolimab alone, 225Ac-trastuzumab alone, and the combination of magrolimab and 225Ac-trastuzumab in an NGS mouse xenograft model using the SK-OV3 human ovarian cancer cell line.

FIG. 2 is a graph showing the comparative effects on tumor growth of vehicle (control), magrolimab alone, ¹⁷⁷Lu-trastuzumab alone, and the combination of magrolimab and ¹⁷⁷Lu-trastuzumab in an NGS mouse xenograft model using the SK-OV3 human ovarian cancer cell line.

FIG. 3 is a graph showing the comparative effects on phagocytosis by human macrophages of BxPC3 human pancreatic cancer cell line (adenocarcinoma) cells of: a non-radiolabeled anti-human HER3 IgG monoclonal antibody AT-02 alone (“HER3 mAb”), an anti-human CD47 antibody alone (10 μg/mL; Clone B6.H12; BioXcell catalog no. BE0019-1; “CD47 mAb”), ²²⁵Ac-labeled AT-02 anti-HER3 mAb alone (100 nCi/mL; ²²⁵Ac-HER3 mAb), and the combination of the anti-CD47 mAb (10 μg/mL) and ²²⁵Ac-labeled AT-02 anti-HER3 mAb (100 nCi/mL). As shown in the figure, the combination prominently enhanced phagocytosis of BxPC3 cells versus any of the individual agents.

FIGS. 4A and 4B are graphs showing that ²²⁵Ac-labeled lintuzumab induces an increase in cell surface calreticulin in human leukemia cell lines.

FIGS. 5A, 5B, and 5C are graphs showing that combination treatment with ²²⁵Ac-labeled lintuzumab and an anti-CD47 antibody enhances phagocytosis of three human leukemia cell lines versus either agent alone.

### DETAILED DESCRIPTION

In one aspect, the presently disclosed invention provides methods for treating a proliferative disease or disorder, such as a hematological malignancy or solid cancer, by administering an effective amount of a radiotherapeutic and an effective amount of a CD47 blockade.

According to certain aspects, the radiotherapeutic may be a radiolabeled targeting agent, such as but not limited to a radiolabeled monoclonal antibody, radiolabeled antigen-binding fragment of a monoclonal antibody, radiolabeled antibody mimetic, radiolabeled peptide or radiolabeled small molecule, that specifically binds to one or more cancer-associated antigens such as the mammalian, for example human, forms of CD33, DR5, 5T4, HER2 (ERBB2; Her2/neu), HER3, TROP2, mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), LYPD3 (C4.4A), Nectin-4, urokinase plasminogen activator receptor (uPAR), Folate receptor alpha (FOLR1), CUB-domain containing protein 1 (CDCP1), Glypican-3 (GPC3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H3, KIT, IL-13Ra2, interleukin-11 receptor a (IL-1 1Ra), PSCA, PRSS21, VEGFR2, LewisY, CD24, platelet-derived growth factor receptor-beta (PDGFR-beta), SSEA-4, CD20, Folate receptor alpha (FRa), MUC1, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECi2A, BST2, EMR2, LY75, GPC3, FCRL5, GPA7, and IGLL1.

According to certain aspects, the CD47 blockade may include a CD47 blocking moiety, such as an antibody against CD47.

Each therapy regime may be administered according to a specific dosing schedule, wherein the method provides for administration of each of the radiotherapy and the CD47 blockade sequentially or simultaneously.

### Definitions and Abbreviations

Throughout this description and in the appended claims, use of the singular includes the plural and plural encompasses singular, unless specifically stated otherwise. For example, although reference is made herein to “an” antibody, “a” radionuclide, and “the” targeting agent, one or more of any of these components and/or any other components described herein may be used.

The words “comprising” and forms of the word “comprising” as well as the word “including” and forms of the word “including,” as used in this description and in the claims, do not limit the inclusion of elements beyond what is referred to. Additionally, although throughout the present disclosure various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled targeting agent, corresponding methods instead reciting “consisting essentially of” or “consisting of” administering the radiolabeled target are also within the scope of said aspects and disclosed by this disclosure.

The term “about” when used in this disclosure in connection with a numerical designation or value, e.g., in describing temperature, time, amount, and concentration, including in the description of a range, indicates a variance of +10% and, within that larger variance, variances of +5% or +1% of the numerical designation or value.

As used herein, “administer”, with respect to a targeting agent such as an antibody, antibody fragment, Fab fragment, aptamer, peptide, or small molecule means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein. For example, according to certain aspects, the targeting agent may be administered as a patient specific therapeutic composition which may be included in a single dose container, the total volume of which may be administered to a patient in a single treatment session. The composition may include a monoclonal antibody or antibody fragment and a pharmaceutically acceptable carrier, wherein a dose of an effector molecule (e.g., radionuclide) of the monoclonal antibody and a total protein amount of the monoclonal antibody may depend on at least one patient specific parameter. Patient specific parameters include, but are not limited to, a patient weight, a patient age, a patient height, a patient gender, a patient medical condition, and a patient medical history.

In addition, compositions including a radiolabeled targeting agent, such as a radiolabeled antibody or radiolabeled antigen-binding antibody fragment, may include one or more pharmaceutically acceptable carriers or pharmaceutically acceptable excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's). An exemplary formulation may be as substantially described in International Pub. No. WO 2017/155937, incorporated by reference herein. For example, according to certain aspects, the formulation may include 0.5% to 5.0% (w/v) of an excipient selected from the group consisting of ascorbic acid, polyvinylpyrrolidone (PVP), human serum albumin (HSA), a water-soluble salt of HSA, and mixtures thereof. Certain formulations may include 0.5-5% ascorbic acid; 0.5-4% polyvinylpyrrolidone (PVP); and the monoclonal antibody in 50 mM PBS buffer, pH 7.

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments or versions thereof, such as Fab, di-Fab, scFvs, diabodies, minibodies, and nanobodies (sdAb); (d) naturally occurring and non-naturally occurring, such as wholly synthetic antibodies, IgG-Fc-silent, and chimeric; and (e) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies may be human, humanized or nonhuman. When a specific aspect of the presently disclosed invention refers to or recites an “antibody,” it is envisioned as referring to any of the full-length antibodies or fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “complementarity-determining region”, or “CDR”, refers to amino acid sequences that, together, define the binding affinity and specificity of the variable region of a native immunoglobulin binding site. There are three CDRs in each of the light and heavy chains of an antibody.

A “framework region”, or “FR”, refers to amino acid sequences interposed between CDRs, typically conserved, that act as the scaffold between the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chains of various subclasses (such as the IgG subclass of heavy chains) are mainly responsible for different effector functions.

As used herein, “Immunoreactivity” refers to a measure of the ability of an immunoglobulin to recognize and bind to a specific antigen. “Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

As used herein, the term “CD33 targeting agent” includes, for example, an antibody, antibody fragment, antibody mimetic, peptide, Fab fragment, aptamer, or small molecule that binds to any available epitope of CD33. According to certain aspects, the anti-CD33 targeting agent is a humanized antibody against CD33, such as lintuzumab (HuM195), gemtuzumab, or vadastuximab. According to certain aspects, the anti-CD33 targeting agent binds to the epitope recognized by the monoclonal antibody “lintuzumab” or “HuM195.” HuM195 is known, as are methods of making it.

As used herein, the term “DR5 targeting agent” includes, for example, an antibody, antibody fragment, antibody mimetic peptide, Fab fragment, aptamer, or small molecule that binds to any available epitope of DR5. According to certain aspects, the anti-DR5 antibody is a human or humanized antibody against DR5. According to certain aspects, the anti-DR5 antibody binds to an epitope of DR5 recognized by the any of mapatumumab, conatumumab, lexatumumab, tigatuzumab, drozitumab, and LBY-135. According to certain aspects, the anti-DR5 antibody is selected from mapatumumab, conatumumab, lexatumumab, tigatuzumab, drozitumab, and/or LBY-135.

TABLE 1

| Company Name | | Product | |
|---|---|---|---|
| (Originator) | Partners | Name | Agent |
| Active Biotech AB | NeoTX | naptumomab estafenatox | Antibody (Fab)- |
| | Therapeutics | (ABR-217620, second | enterotoxin fusion |
| | Ltd. | generation) | protein |
| Aptevo Therapeutics | Alligator | ALG.APV-527 | Antibody (dual |
| Inc. | Bioscience AB | | targeting) |
| Asana BioSciences | Mersana | ASN004 | Antibody-drug |
| LLC | | | conjugate; scFvFc |
| | | | structure |
| Biotecnol Ltd. | Chiome | Tb535 | Antibody (triabody) |
| | Bioscience | | |
| Ambrx Inc. | Pfizer Inc. | Anti-5T4 ADC | Antibody-drug |
| | | | conjugate |
| Byondis | Previous name: | Anti-5T4 SyD1875 | Antibody-drug |
| | Synthon | | conjugate |
| Genmab | Abbvie | GEN1044 (DuoBody- | Bispecific mAb |
| | | CD3 × 5T4) | |
| Guangdong | — | H6-DM4 | Antibody-drug |
| Zhongsheng | | | conjugate |
| Pharmaceutical Co | | | |
| Medimmune | AstraZeneca | MEDI0641 | Antibody-drug |
| | | | conjugate |
| Oxford Biomedica | — | H8 | Antibody |
| Pfizer Inc. | Oxford | PF-06263507/ | Antibody-drug |
| | Biomedica | A1mcMMAF | conjugate |
| Zova | Neoantigen | ZV0508 | Antibody-drug |
| Biotherapeutics | Therapeutics | | conjugate |
| Macrogenics | — | 5T4 × CD137 TRIDENT | Bispecific mAb |
| Macrogenics | — | 5T4 × CD3 DART ® | Bispecific mAb |
| Amgen | — | 5T4-CD3 Bispecific | Bispecific mAb |

As used herein, the term “5T4 targeting agent” includes, for example, an antibody, antibody fragment, antibody mimetic, peptide, Fab fragment, aptamer, or small molecule that binds to any available epitope of 5T4. For example, the 5T4 targeting agent may be a monoclonal antibody. The original description of an anti-5T4 antibody sequence was provided by Hole & Stem (Hole & Stern (1988) Br. J. Cancer 57, 239-246). An antibody for use as an 5T4 targeting agent according to the presently disclosed invention, such as in preclinical studies, may be produced using the sequence provided by Hole & Stern. According to certain aspects, the 5T4 targeting agent is a humanized antibody against 5T4, such as described in U.S. Pat. Nos. 7,074,909 and 8,044,178. Exemplary antibodies against 5T4 include at least MED10641, described in Harper (Harper, J. et al. (2017) Mol. Cancer Ther. 16, 1576-1587) and developed by Medimmune/AstraZeneca; ALG.APV-527, developed by Aptevo Therapeutics/Alligator Bioscience; Tb535, developed by Biotecnol/Chiome Bioscience; H6-DM5 developed by Guangdong Zhongsheng Pharmaceuticals; and ZV0508 developed by Zova Biotherapeutics. See also Table 1 disclosing further antibodies and antibody-drug conjugates, wherein the anti-5T4 portions thereof may be used as 5T4 targeting agents in the various aspects of the presently disclosed invention.

As used herein, an “anti-HER2 antibody” is an antibody, such as but not limited to a monoclonal antibody (mAb), that binds to any available epitope of HER2 (ErbB2). According to certain aspects, the anti-HER2 antibody employed may be Trastuzumab or a different antibody that binds to an epitope of HER2 recognized by Trastuzumab and/or the antibody employed may be Pertuzumab or a different antibody that binds to an epitope of HER2 recognized by Pertuzumab. According to certain aspects, the anti-HER2 antibody may also be a multispecific antibody, such as bispecific antibody, against any available epitope of HER3/HER2 such as MM-111 and MM-141/Istiratumab from Merrimack Pharmaceuticals, MCLA-128 from Merus NV, and MEHD7945A/Duligotumab from Genentech.

The amino acid sequences of the light chain and the heavy chain of Trastuzumab reported by DrugBank Online are: light chain (SEQ ID NO:102) and heavy chain (SEQ ID NO:103).

Applicants have successfully conjugated Trastuzumab with p-SCN-DOTA and radiolabeled the composition with ¹²⁵Ac or ¹⁷⁷Lu.

The amino acid sequences of the light chain and the heavy chain of Pertuzumab reported by DrugBank Online are: light chain (SEQ ID NO:104) and heavy chain (SEQ ID NO:105).

Still other radiolabeled HER2-targeting agents that may be used or embodied in the various aspects of the invention include ²¹²Pb-TCMC-Trastuzumab (Orano Med) and ¹³¹I-CAM-H2 (¹³¹-Iodine conjugated anti-HER2 sdAb 2Rs15d; Precirix NV) to treat HER2 expressing cancers, such as breast cancers, advanced/metastatic HER2-positive breast cancer, gastric cancer, gastro-esophageal junction (GEJ) cancer and any of those disclosed herein.

As used herein, an “anti-HER3 antibody” is an antibody, such as but not limited to a monoclonal antibody (mAb), that binds to any available epitope of HER3. According to certain aspects, the anti-HER3 antibody may be one of the following antibodies or bind to an epitope of HER3 recognized by one of the following antibodies: Patritumab, Seribantumab, Lumretuzumab, Elgemtumab, AV-203 (a/k/a CAN017; Aveo Oncology), or GSK2849330. According to certain aspects, the anti-HER3 antibody is selected from one or more of Patritumab, Seribantumab, Lumretuzumab, Elgemtumab, US-1402, AV-203, CDX-3379, or GSK2849330. According to certain aspects, the anti-HER3 antibody may be a multispecific antibody, such as a bispecific antibody, against any available epitope of HER3/HER2 such as MM-111 and MM-141/Istiratumab from Merrimack Pharmaceuticals, MCLA-128 from Merus NV, and MEHD7945A/Duligotumab from Genentech. The antibody may, for example, be one of the anti-HER3 antibodies disclosed in U.S. Pub No. 20210025006 such as CAN017 (heavy chain SEQ ID NO:119 and light chain SEQ ID NO:120), 04D01 (heavy chain SEQ ID NO:121 and light chain SEQ ID NO:122), 09D03 (heavy chain SEQ ID NO:123 and light chain SEQ ID NO:124), 11 G01 (heavy chain SEQ ID NO:125 and light chain SEQ ID NO:126), 12A07 (heavy chain SEQ ID NO:127 and light chain SEQ ID NO:128), 18H02 (heavy chain SEQ ID NO:129 and light chain SEQ ID NO:130) and 22A02 (heavy chain SEQ ID NO:131 and light chain SEQ ID NO:132), an IgG having the heavy chain of SEQ ID NO:133 and the light chain of SEQ ID NO:134, a HER3-binding antibody, such as an IgG, having a heavy chain including 1, 2 or 3 of the heavy chain CDRs of any of said antibodies and/or having a light chain having 1, 2 or 3 of the light chain CDRs of said antibodies, or an antibody binding to an epitope of HER3 recognized by any of said antibodies.

An “epitope” refers to the target molecule site (e.g., at least a portion of an antigen) that is capable of being recognized by, and bound by, a targeting agent such as an antibody, antibody fragment, Fab fragment, aptamer, or small molecule. For a protein antigen, for example, this may refer to the region of the protein (i.e., amino acids, and particularly their side chains) that is bound by the targeting agent. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, the terms “proliferative disorder” and “cancer” may be used interchangeably and may include, without limitation, a solid cancer (e.g., a tumor). “Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

According to certain aspects, the solid cancer may be breast cancer such as tamoxifen-sensitive breast cancer, tamoxifen-resistant breast cancer or triple negative breast cancer (TNBC), gastric cancer, bladder cancer, cervical cancer, endometrial cancer, skin cancer such as melanoma, stomach cancer, testicular cancer, esophageal cancer, bronchioloalveolar cancer, prostate cancer such as castration resistant prostate cancer (CRPC), colorectal cancer, ovarian cancer, cervical epidermoid cancer, liver cancer such as hepatocellular carcinoma (HCC) or cholangiocarcinoma, pancreatic cancer, lung cancer such as non-small cell lung carcinoma (NSCLC) or small cell lung cancer (SCLC), renal cancer, head and neck cancer such as head and neck squamous cell cancer, a carcinoma, a sarcoma, or any combination thereof.

As used herein, “cancer” also includes, without limitation, a hematologic malignancy. A “hematologic disease” or “hematological disorder” may be taken to refer to at least a blood cancer. Such cancers originate in blood-forming tissue, such as the bone marrow or other cells of the immune system. A hematologic disease or disorder includes, without limitation, leukemias (such as acute myeloid leukemia (AML), acute promyelocytic leukemia, acute lymphoblastic leukemia (ALL), acute mixed lineage leukemia, chronic myeloid leukemia (CMIL), chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma (HL), non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

According to certain aspects, the radiotherapeutic may include a targeting agent labeled with a radioisotope. As used herein, a “radioisotope” and “radionuclide” may be used interchangeably, and can be an alpha-emitting isotope, a beta-emitting isotope, and/or a gamma-emitting isotope. Examples of radioisotopes include the following: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra ²²⁷Th, ¹⁴⁹Tb, ¹³⁷Cs, ²¹²Pb and ¹⁰³Pd. Methods for affixing a protein such as an antibody or antibody fragment (i.e., “labeling” an antibody with a radioisotope) are well known. Specific methods for labeling are described, for example, in U.S. Pat. No. 9,603,954, International Publication No. WO 2017/155937 and U.S. Provisional Patent Application No. 63/119,093 filed Nov. 30, 2020 and titled “Compositions and methods for preparation of site-specific radioconjugates,” both of which are incorporated by reference herein.

For example, according to certain aspects, the radiotherapeutic targeting agent may be labeled by (a) conjugating a targeting agent such as an antibody or peptide with a chelant, such as p-SCN-Bn-DOTA, in a buffered solution, (b) labeling the chelant-conjugated targeting agent with a radionuclide in a buffered solution, such as 225-Actinium or “²²⁵Ac”, (c) quenching the reaction by the addition of a quenching chelate (e.g. diethylenetriaminepentaacetic acid (DTPA)), and (d) purifying the radiolabeled chelator-conjugated targeting agent, for example, via filtration. Exemplary chelators include compounds having the dual functionality of sequestering metal ions, such as the radionuclide, plus the ability to covalently bind a biological carrier/targeting agent such as an antibody.

Exemplary chelators that may be used include, but are not limited to S-2-(4-Isothiocyanatobenzyl)-1,4,7,10 tetraazacyclododecanetetraacetic acid (p-SCN-Bn-DOTA), diethylene triamine pentaacetic acid (DTPA); ethylene diamine tetraacetic acid (EDTA); 1,4,7,10-tetra-azacyclododecane-N,N′,N″,N′″-tetraacetic acid (DOTA); p-isothiocyanatobenzyl-1,4,7,10-tetra-azacyclododecane-1,4,7,10-te-traacetic acid (p-SCN-Bz-DOTA); 1,4,7,10-tetra-azacyclododecane-N,N′,N″-triacetic acid (DO3A); 1,4,7,10-tetra-azacyclododecane-1,4,7,10-tetrakis(2-propionic acid) (DOTMA); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (NOTA); 1,4,8,11-tetra-azacyclotetradecane-N,N′,N″,N′″-tetraacetic acid (TETA); triethylene tetraamine hexaacetic acid (TTHA); trans-1,2-diaminohexane tetraacetic acid (CYDTA); 1,4,7,10-tetra-azacyclododecane-1-(2-hydroxypropyl)-4,7,10-triacetic acid (HP-D03A); trans-cyclohexane-diamine tetraacetic acid (CDTA); trans(1,2)-cyclohexane dietylene triamine pentaacetic acid (CDTPA); 1-oxa-4,7,10-triazacyclododecane-N,N′,N″-triacetic acid (OTTA); 1,4,7,10-tetra-azacyclododecane-1,4,7,10-tetrakis{3-(4-carboxyl)-butanoic acid}; 1,4,7,10-tetra-azacyclododecane-1,4,7,10-tetrakis(acetic acid-methyl amide); 1,4,7,10-tetra-azacyclododecane-1,4,7,10-tetrakis(methylene phosphonic acid); and derivatives thereof.

According to certain aspects, when the radiotherapeutic targeting agent is ²²⁵Ac-labeled, the effective amount is below 50 μCi/kg, 40 μCi/kg, 30 μCi/kg, 20 μCi/kg, 10 μCi/kg, 5 Ci/kg, 4 μCi/kg, 3 μCi/kg, 2 μCi/kg, 1 μCi/kg, or even 0.5 μCi/kg. According to certain aspects, the effective amount is at least 0.05 μCi/kg, or 0.1 μCi/kg, 0.2 μCi/kg, 0.3 μCi/kg, 0.4 μCi/kg, 0.5 Ci/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, Ci/kg, 12 μCi/kg, 14 μCi/kg, 15 μCi/kg, 16 μCi/kg, 18 μCi/kg, 20 μCi/kg, 30 μCi/kg, or 40 Ci/kg. According to certain aspects, the ²²⁵Ac-labeled targeting agent may be administered at a dose that includes any combination of upper and lower limits as described herein, such as from at least 0.1 μCi/kg to at or below 5 μCi/kg, or from at least 5 μCi/kg to at or below 20 μCi/kg.

According to certain aspects, the radiotherapeutic targeting agent is ²²⁵Ac-labeled, and the effective amount may be below 2 mCi (i.e., wherein the ²²⁵Ac is administered to the subject in a non-weight-based dosage). According to certain aspects, the effective amount may be below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi, 20 μCi, 10 μCi, or 5 μCi. The effective amount may be at least 2 μCi, such as at least 5 μCi, 10 μCi, 20 μCi, 30 μCi, 40 μCi, 50 μCi, 60 Ci, 70 μCi, 80 μCi, 90 μCi, 100 μCi, 200 μCi, 300 μCi, 400 μCi, 500 μCi, 600 μCi, 700 μCi, 800 Ci, 900 μCi, 1 mCi, 1.1 mCi, 1.2 mCi, 1.3 mCi, 1.4 mCi, or 1.5 mCi. According to certain aspects, the ²²⁵Ac-labeled CD33 targeting agent may be administered in an amount that includes any combination of upper and lower limits as described herein, such as from at least 2 μCi to at or below 1 mCi, or from at least 2 μCi to at or below 250 μCi, or from 75 μCi to at or below 400 μCi.

According to certain aspects, the ²²⁵Ac-labeled radiotherapeutic targeting agent includes a single dose that delivers less than 12Gy, or less than 8 Gy, or less than 6 Gy, or less than 4 Gy, or less than 2 Gy, such as doses of 2 Gy to 8 Gy, to the subject, such as predominantly to the targeted solid tumor.

According to certain aspects, the radiotherapeutic targeting agent is radiolabeled with ¹⁷⁷Lu (“¹⁷⁷Lu-labeled”), and the effective amount may be, for example, below 1 mCi/kg (i.e., where the amount of ¹⁷⁷Lu-labeled targeting agent administered to the subject delivers a radiation dose of below 1000 mCi per kilogram of subject's body weight). According to certain aspects, the effective amount is below 900 μCi/kg, 800 μCi/kg, 700 μCi/kg, 600 μCi/kg, 500 μCi/kg, 400 Ci/kg, 300 μCi/kg, 200 μCi/kg, 150 μCi/kg, 100 μCi/kg, 80 μCi/kg, 60 μCi/kg, 50 μCi/kg, 40 Ci/kg, 30 μCi/kg, 20 μCi/kg, 10 μCi/kg, 5 μCi/kg, or 1 μCi/kg. According to certain aspects, the effective amount is at least 1 μCi/kg, 2.5 μCi/kg, 5 μCi/kg, 10 μCi/kg, 20 μCi/kg, 30 μCi/kg, 40 Ci/kg, 50 μCi/kg, 60 μCi/kg, 70 μCi/kg, 80 μCi/kg, 90 μCi/kg, 100 μCi/kg, 150 μCi/kg, 200 Ci/kg, 250 μCi/kg, 300 μCi/kg, 350 μCi/kg, 400 μCi/kg or 450 μCi/kg. According to certain aspects, an ¹⁷⁷Lu-labeled targeting agent may be administered in an amount that includes any combination of upper and lower limits as described herein, such as from at least 5 mCi/kg to at or below 50 μCi/kg, or from at least 50 mCi/kg to at or below 500 μCi/kg.

According to certain aspects, the radiotherapeutic targeting agent is ¹⁷⁷Lu-labeled, and the effective amount may be below 45 mCi, such as below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 μCi. According to certain aspects, the effective amount may be at least 10 μCi, such as at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. According to certain aspects, an ¹⁷⁷Lu-labeled targeting agent may be administered in an amount that includes any combination of upper and lower limits as described herein, such as from at least 10 mCi to at or below 30 mCi, or from at least 100 μCi to at or below 3 mCi, or from 3 mCi to at or below 30 mCi.

According to certain aspects, the radiotherapeutic targeting agent is radiolabeled with ¹³¹I (“¹³¹I-labeled”), and the effective amount may be below, for example, 1200 mCi (i.e., where the amount of ¹³¹I administered to the subject delivers a total body radiation dose of below 1200 mCi in a non-weight-based dose). According to certain aspects, the effective amount may be below 1100 mCi, below 1000 mCi, below 900 mCi, below 800 mCi, below 700 mCi, below 600 mCi, below 500 mCi, below 400 mCi, below 300 mCi, below 200 mCi, below 150 mCi, or below 100 mCi. According to certain aspects, the effective amount may be below 200 mCi, such as below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. According to certain aspects, the effective amount may be at least 1 mCi, such as at least 2 mCi, 3 mCi, 4 mCi, 5 mCi, 6 mCi, 7 mCi, 8 mCi, 9 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. According to certain aspects, an ¹³¹I-labeled targeting agent may be administered in an amount that includes any combination of upper and lower limits as described herein, such as from at least 1 mCi to at or below 100 mCi, or at least 10 mCi to at or below 200 mCi.

While select radionuclides have been disclosed in detail herein, any of those disclosed herein are contemplated for labeling the targeting agents (i.e., radiotherapeutic or radioimmunotherapy) that are part of the presently disclosed invention.

According to certain aspects of the presently disclosed invention, a majority of the radiotherapeutic targeting agent (antibody, antibody fragment, peptide, small molecule, etc.) administered to a subject typically consists of non-labeled targeting agent, with the minority being the labeled targeting agent. The ratio of labeled to non-labeled targeting agent can be adjusted using known methods. According to certain aspects, the radiotherapeutic (e.g., radioimmunotherapy) may include a labeled fraction and an unlabeled fraction, wherein the ratio of labeled:unlabeled may be from about 0.01:10 to 1:1, such as 0.1:10 to 1:1 labeled:unlabeled. Moreover, the radiotherapeutic may be provided as a single dose composition tailored to a specific patient, wherein the amount of labeled and unlabeled targeting agent in the composition may depend on at least a patient weight, age, gender, diagnosis, and/or disease state or health status, such as detailed in International Pub. No. WO 2016/187514.

This inventive combination of a labeled fraction and a non-labeled fraction of the targeting agent of the radiotherapeutic allows the composition to be tailored to a specific patient. For example, when the radiotherapeutic is a radioimmunotherapy (i.e., the targeting agent is an antibody), each of the radiation dose and the protein dose of the antibody may be personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of the disclosed composition is that there will be no left-over radiation that would need to be discarded or handled by the medical personnel, e.g., no dilution, or other manipulation to obtain a dose for the patient. When provided in a single dose container, the container may simply be placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

According to certain aspects, when the radiotherapeutic targeting agent is an antibody, it may be provided in a total protein amount of up to 100 mg, such as up to 60 mg, such as 5 mg to 45 mg, or a total protein amount of between 0.01 mg/kg patient weight to 16.0 mg/kg patient weight, such as between 0.01 mg/kg patient weight to 10.0 mg/kg, or between 0.05 mg/kg patient weight to 5.0 mg/kg, or between 0.01 mg/kg patient weight to 1.0 mg/kg, or between 0.01 mg/kg patient weight to 0.6 mg/kg patient weight, or 0.01 mg/kg patient weight, 0.015 mg/kg patient weight, 0.02 mg/kg patient weight, or 0.04 mg/kg patient weight, or 0.06 mg/kg patient weight.

According to certain aspects, the effective amount of an antibody in the radioimmunotherapy may include a total protein amount of less than 10 mg/m², such as about 6 mg/m², or 3 mg/m², or even 2 mg/m².

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject may, for example, be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells). It should be understood that wherever in this disclosure a cancer-associated target antigen is disclosed, the invention provides methods for treating a cancer, whether hematological or solid, that expresses or overexpresses said target antigen, which method includes administering a radiolabeled targeting agent that binds said target antigen to a mammalian subject such as a human patient, in need of treatment for the cancer in combination with or in conjunction administration of one or more CD47 blockades to the subject.

“Therapeutically effective amount” or “effective amount” refers to an amount effective, at dosages and for periods of time necessary, to achieve a desired therapeutic result. A therapeutically effective amount may vary according to factors such as the disease state, age, gender, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the therapeutic agent, i.e., radiotherapeutic or CD47 blockade that may deplete or cause a reduction in the overall number of cancer cells, such as a reduction in certain hematological cells (e.g., CD33 expressing cells), or DR5 expressing cells, or 5T4 expressing cells, HER2, or HER3 expressing cells, or TROP2 expressing cells or may inhibit growth of a tumor, when used together or when used separately.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

Throughout this application, various patents, patent applications and other publications are cited. The disclosures of these patents, patent applications and other publications are hereby incorporated by reference in their entireties into this application.

Unless otherwise defined or clear from the context in which presented, technical and scientific terms used herein have the same meaning as commonly understood by one of ordinary skill in the art to which the presently disclosed invention belongs. Although methods and materials similar or equivalent to those described herein may be used in the practice or testing described herein, suitable methods and materials are described below.

### Aspects of the Invention

The present disclosure relates to methods for treating a mammalian subject, such as a human patient, with cancer by administration of a radiotherapeutic and a CD47 blockade. The radiotherapeutic may include a radiolabeled cancer targeting agent, such as a radiolabeled antibody that recognizes a cancer-associated antigen, and the CD47 blockade may include an agent that prevents CD47 binding to SIRPα, such as an anti-CD47 blocking antibody or affinity agent or an anti-SIRPα blocking antibody, or an agent that otherwise downregulates CD47-SIRPα axis activity.

CD47 (originally named integrin-associated protein (IAP)) is a cell surface protein of the immunoglobulin (Ig) superfamily, which is heavily glycosylated and expressed by virtually all cells in the body. Typically associated with integrin avb3 on most cell types, except RBCs (which lack integrins), it is an indicator of self, providing a “don't eat me signal” to macrophages/phagocytes. That is, cell-surface CD47 interacts with its receptor on macrophages, SIRPα, to inhibit phagocytosis of normal, healthy cells. CD47 is upregulated on circulating hematopoietic stem cells and leukemia cells to avoid phagocytosis. CD47 is also highly expressed on several human cancers including myelodysplastic syndrome (MDS), acute myeloid leukemia (AML), non-Hodgkin lymphoma, and bladder cancer as a means to evade phagocytosis by the innate immune system (Eladl, et al. (2020) Hematology & Oncology, 13:96).

There are as many as thirty CD47 blocking agents being developed for the treatment of cancer in both solid tumors and hematological malignancies. Strategies to block the CD47-SIRPα axis include biologics that bind or otherwise affect either CD47 or SIRPα and many are currently in clinical testing. Examples include magrolimab, lemzoparlimab, and AO-176. Although these molecules block the engagement between CD47 and SIRPα, they may be engineered to include or ablate Fc function, e.g., IgG1 vs IgG2 or IgG4, so the mechanistic properties of these molecules may be different. For example, certain CD47 blocking antibodies also bind to CD47 expressed on red blood cells, and as a result, in the clinic, a related adverse event is anemia.

Anti-tumor responses have been observed in preclinical trials, such as for the anti-CD47 antibody AO-176, and in clinical human trials, however the overall response to single agent anti-CD47 or anti-SIRPα has been modest. This is likely due to the necessity for up-regulation of pro-phagocytic responses, e.g., eat me signals, in addition to don't eat me blockade to enable efficient tumor cell phagocytosis. Under normal physiologic conditions, cellular homeostasis is partly regulated by balancing pro- and anti-phagocytic signals. For target cells to be phagocytosed upon CD47 blockade, the cells must also display a potent pro-phagocytic signal, the main “eat me” signals being elicited by surface expressed calreticulin and phosphatidylserine. It is therefore likely that drugs that target the CD47-SIRPα axis will require therapeutic combinations to enable significant clinical responses.

The presently disclosed invention relates directly to compositions and methods that tip the balance of cellular homeostasis toward pro-phagocytic signals, such as for specific cell types involved in cancers and hematological malignancies. To this end, the presently disclosed invention relates to a blockade of the CD47 interaction with SIRPα (on phagocytic cells) that interrupts or otherwise downregulates the “don't eat me” signal, in combination with a radiotherapeutic that enhances the “eat me” signal.

As an example, the anti-CD47 antibody magrolimab recently demonstrated significant clinical responses in high risk previously untreated patients with myelodysplastic syndrome (MDS) or acute myeloid leukemia (AML). The requirement for up-regulation of eat me signals appears critical to enable these responses. Radiation is an ideal combination therapy for agents that block the CD47 pathway due to its ability to induce both the innate and adaptive immune response (de la Cruz-Merino, et al. (2014) Frontiers in Immunology, vol 5, article 102; Vermeer, et al. (2012) International Journal of Cancer, 133:120).

The radiobiological response causes the activation of different T-cell lines, generating the “switch-on” of the adaptive immune response. The radiobiological model considers that DNA damage after radiation induces different types of biological response as a result of direct damage of tumor cells or indirectly due to induction of free radicals. Most cells survive for a limited time after irradiation and, during this time, they generate molecular signals (damage-associated molecular patterns (DAMPs)) that induce the overexpression of specific genes that control the expression of growth factors, cytokines, chemokines, and cell surface receptors —activate both innate and adaptive immune system inflammatory response.

Radiation has been delivered to cells via IR and to human patients via directed external beam radiation. This radiation has been found to enhance cancer-specific peptide release from damaged cancer cells, facilitate antigen uptake and presentation by dendritic cells, decrease CD47 and increases calreticulin, and upregulate MHC-I expression on tumor cells to increase cancer cell recognition by T cells. Moreover, the radiation-induced DNA damage triggers cGAS-STING pathway to activate IFN gene transcription.

However, this modality is not an option for patients with hematologic, or blood, cancers since their disease is disseminated and systemic irradiation by external beam radiation would expose normal tissues and organs to radiation, risking significant toxicities. To the contrary, targeted radiation with an antibody radioconjugate (ARC), or other radiotherapeutic directed to a selective tumor antigen or otherwise preferentially targeting cancer cells enables the effective delivery of the potency of radiation to the tumor cells by the targeting agent. Such a combination leads to in vivo tumor elimination in targeted cells without significant effects on most normal cells.

Exemplary radiotherapeutics of the presently disclosed invention include antibody radioconjugates (ARCs) against CD33, DR5, 5T4, HER2, HER3 and/or TROP2. Exemplary ARCs include any of an anti-CD33, DR5, 5T4, HER2, HER3 and/or TROP2 antibody labeled with the potent alpha particle emitting radioisotope actinium-225 (²²⁵Ac). For example, when the radiotherapeutic includes an actinium-225 labeled monoclonal antibody against CD33, such as ²²⁵Ac-lintuzumab, the radiation is delivered directly to CD33 positive cells and finds use as a therapeutic against heme malignancies such as AML, MDS, and multiple myeloma. By delivering radiation directly to tumor cells, ARCs have the potential to affect the potent radiobiologic effects of external beam radiation in a manner safe for administration to patients, and especially those with a disseminated disease. As a result, exposure of tumor cells to the ARC's of the present invention will up-regulate ‘eat me’ signals such as calreticulin and down-regulate CD47 on the surface of cancer cells.

As such, the combination use of an ARC with a CD47-SIRPα blocking agent is an object of the present invention and enhances the pro-phagocytic response to a CD47-SIRPα blockade as a result of the radioimmunobiologic effects of the targeted radionuclide warhead.

Further, since targeted ARC radiation itself can impart a direct anti-tumor effect, as well as further stimulate the adaptive immune response, the combination of these two types of agents provides a synergistic therapeutic, improving both the therapeutic outcomes and durability of the response. For example, ²²⁵Ac-lintuzumab has demonstrated evidence of clinical activity and tolerability in human trials in relapsed/refractory AML and has shown promising responses in early combination studies with standard of care therapies. Several anti-CD47 blocking agents are currently being tested as single agent and in combination with chemotherapy and targeted therapy in myeloid diseases such as AML and MDS. The combination of ²²⁵Ac-lintuzumab with a CD47 blocking agent in myeloid diseases provides a potent and potentially well-tolerated therapeutic strategy in these diseases. This approach can also be extended to other tumor types including solid tumors and other blood cancers.

Radiotherapeutic Agents Targeting CD33

The overexpression of CD33 is commonly found in hematological malignancies, including AML, CML, and MDS. In AML, 85-90% of patients express CD33, which has led to the development of targeted therapies, such as gemtuzumab-ozogamicin (Mylotarg™). Approximately 96% of MDS patients express CD33 on their myeloblasts (Sanford et al. (2016) Leukemia & Lymphoma, vol. 57(8):1965-1968). In another study, MDS patients demonstrated approximately twice as many CD33 molecules per bone marrow cell as the control samples (Jilani, et al. (200) Am J Clin Pathol vol. 118:560-566). The CD33 antigen is expressed on virtually all cases of CML. Moreover, patients older than 60 years have a poor prognosis with only 10% to 15% of 4-year disease-free survival for AML. This high relapse rate for AML patients and the poor prognosis for older patients highlight the urgent need for novel therapeutics preferentially targeting CD33⁺ cells.

Accordingly, the methods disclosed herein may include administration of a radioimmunotherapy against CD33 in combination with a CD47 blockade. Such methods may be used to treat a proliferative disorder such as a solid cancer and/or a hematological disease or disorder (e.g., a hematological cancer) and/or may be used to inhibit growth and/or proliferation of a cell expressing CD33, and/or may also be used to treat a disease or disorder involving cells expressing or overexpressing CD33. Moreover, the methods may be used to treat a hematological disease or disorder which is multiple myeloma, acute myeloid leukemia, myelodysplastic syndrome, myeloproliferative neoplasm, chronic myeloid leukemia (CML), or a relapsed/refractory (relapsed and/or refractory) form of any of the preceding.

Radiolabeled CD33 targeting agents may also be used to deplete CD33-expressing myeloid-derived suppressor cells (MDSCs) found in solid tumors or which act as immune suppressors in hematological cancers, such as leukemias and lymphomas, in the treatment of such disorders. Thus, according to one aspect of the invention, one or more radiolabeled CD33 targeting agents, such as a radiolabeled anti-CD33 monoclonal antibody or a radiolabeled CD33-binding antibody fragment, such as an ²²⁵Ac labeled anti-CD33 antibody, such as ²²⁵Ac lintuzumab, may be administered to a mammalian subject, such as a human subject, in need of treatment of a hematological cancer or proliferative disorder or a solid tumor, such as a solid tumor infiltrated by or subject to infiltration by MDSCs, in combination or conjunction with administering one or more CD47 blockades to the subject. Suitable CD33 targeting agents for radiolabeling and use include, for example, lintuzumab, gemtuzumab, vadastuximab, antibodies having the heavy chain and light chain CDRs of the preceding monoclonal antibodies, and CD33-binding fragments of any of the preceding monoclonal antibodies. The solid tumor may, for example, be a sarcoma, osteosarcoma, fibrosarcoma, pancreatic cancer, breast cancer, tamoxifen-resistant breast cancer, TNBC, hepatocellular carcinoma, melanoma, lung cancer, small cell lung cancer (SCLC), non-small cell lung cancer (NSCLC), Lewis lung carcinoma, Solid Ehrlich carcinoma, endometrial cancer, glioma, glioblastoma, mesothelioma, carcinomas, colon cancer, colorectal carcinoma, oral carcinoma, renal carcinoma, ovarian cancer, or any of the solid tumor cancers disclosed herein.

CD33 is a 67 Kd type I transmembrane receptor glycoprotein that may function as a sialic acid-dependent cell adhesion molecule. CD33 has a long N-terminal extracellular domain, a helical transmembrane domain, and a short C-terminal cytoplasmic domain. Expressed on early myeloid progenitor and myeloid leukemic (e.g., acute myelogenous leukemia, AML) cells, CD33 is not expressed on stem cells. Amino acid residues 1-259 of the CD33 protein represent the extracellular domain, amino acids 260-282 represent the helical transmembrane domain, and amino acids 283-364 represent the cytosolic domain (intracellular). There are at least three known single nucleotide polymorphisms (“SNPs”) in the extracellular domain of CD33 (i.e., W22R, R69G, S128N).

Antibodies against CD33, such as lintuzumab (HuM195), gemtuzumab, and vadastuximab have been, and are currently being evaluated in the clinic for their efficacy to treat hematological malignancies and plasma cell disorders, including acute myeloid leukemia (AML). Each antibody has been found to bind to a different portion of the extracellular region of CD33, and each demonstrates different clinical responses (e.g., anti-tumor effects). Gemtuzumab is available from Pfizer as the ADC Mylotarg™, and vadastuximab is available from Seattle Genetics as the ADC Vadastuximab talirine.

Studies with an unconjugated M195, derived from a mouse immunized with live human leukemic myeloblasts, demonstrated transient decreases in peripheral blast counts in human patients when administered at saturating or supra-saturating doses. The humanized antibody HuM195 was constructed by grafting complementarity-determining regions of M195 into a human IgG1 framework and backbone. HuM195 was found to have greater than 8-fold higher binding avidity than M195 and, unlike M195, demonstrated antibody-dependent cell-mediated cytotoxicity (ADCC). Still, while limited studies point toward some activity in acute promyelocytic leukemias (APL) when used in in patients with minimal residual disease, HuM195 has very modest activity as a single agent in AML even at supra-saturating doses that fully blocked CD33 binding sites throughout a 4-week period, with the infrequent achievement of complete or partial remissions limited to patients with low tumor burden. Efficacy could perhaps be increased if supra-saturating doses are given repeatedly, as suggested by a small trial in which very high doses of lintuzumab were given weekly for 5 weeks and then every other week for patients with clinical benefit.

While these currently available anti-CD33 antibodies eliminate CD33-positive cells by antibody-dependent cellular cytotoxicity (ADCC), complement-dependent cytotoxicity (CDC), and apoptosis in their unlabeled state, the currently disclosed invention provides use of such antibodies including a radiolabel (ARCs). As such, the concentrations required to induce the radiobiologic effects on targeted cells, as disclosed herein, are much lower. Thus, the negative side effects associated with the high concentrations of the unlabeled antibodies of the prior art methods are reduced or eliminated.

Moreover, when the anti-CD33 antibodies are radiolabeled with an alpha-emitting radionuclide, such as actinium-225, the effects of the radiobiologic are highly targeted. The ²²⁵Ac payload delivers high energy alpha particles directly to the tumor site or CD33 expressing cells, generating lethal double strand DNA breaks without necessitating significant payload accumulation within the tumor cell, and providing therapeutic efficacy for even low target antigen expressing tumors. Due to its short path length, the range of its high energy alpha particle emission is only a few cell diameters thick, thereby limiting damage to nearby normal tissues. Manufacturing of Lintuzumab Satetraxetan Ac-225 is described, for example, in U.S. Pat. No. 9,603,954.

Radiotherapeutic Agents Targeting DR5

Humans express two functional death receptors (DR4 and DR5), also known as tumor necrosis factor-related apoptosis-inducing ligand receptors 1 and 2 (TRAIL-R1 and -R2), which become upregulated on cell surfaces as part of an immune surveillance mechanism to alert the immune system of the presence of virally infected or transformed cells. TRAIL, the ligand that binds death receptors, is expressed on immune cells such as T-cells and NK cells, and upon engagement of DR4 or DR5, TRAIL trimerizes the death receptor and induces an apoptotic cascade that is independent of p53 (Naoum, et el. (2017) Oncol. Rev. 11, 332). While DR4 and DR5 can be found expressed at low levels in some normal tissues (Spierings, et al. (2004) J. Histochem. Cytochem., 52, 821-31), they are upregulated on the surface of many tumor tissues including renal, lung, acute myeloid leukemia (AML), cervical, and breast cancers.

Following the identification of death receptors as a viable therapeutic target, many DR4 and DR5-targeting antibodies and recombinant TRAIL (rTRAIL) proteins have been developed, including mapatumumab, conatumumab, lexatumumab, tigatuzumab, drozitumab, and LBY-135. Tigatuzumab has been evaluated in a Phase 2 clinical trial in triple negative breast cancer (TNBC) patients, wherein the expression of DR5 on both primary and metastatic tumor samples was confirmed, demonstrating that DR5 is a suitable target for directing therapeutic intervention in this cancer type and metastatic disease (Forero-Torres, et al. (2015) Clin. Cancer Res., 21, 2722-9).

In treatment regimens targeting solid tumors, such as breast cancer, radiation is typically used only to treat the site of the primary tumor after surgical resection and is only used palliatively for metastases. An additional or alternative approach to achieve targeted delivery of radiation to both primary and metastatic tumors and to spare normal tissues from radiation toxicity is through use of a radiotherapeutic, as disclosed herein, directed to the tumor related antigen DR5.

Accordingly, radiotherapeutic agents that may be used include at least antibodies, peptides, and/or small molecules that target DR5. Exemplary radiotherapeutics include ARCs targeted to DR5, such as radiolabeled monoclonal antibodies against DR5 (e.g., ²²⁵Ac-DR5). Exemplary antibodies against DR5 include at least tigatuzumab (CD-1008) from Daiichi Sankyo, conatumumab (AMG 655) from Amgen, mapatumumab from AstraZeneca, lexatumumab (also known as ETR2-ST01) from Creative Biolabs (Shirley, N.Y., USA), LBY-135, and drozitumab from Genentech. Studies in mice may use the surrogate mouse antibody TRA-8 or MD5-1.

Radiotherapeutic Agents Targeting 5T4

Trophoblast glycoprotein (TBPG), also known as 5T4, is a glycoprotein that is categorized as an oncofetal antigen, meaning it is expressed on cells during fetal developmental stages but is not expressed in adult tissues except on tumors (Southall, P. J. et al. (1990) Br. J. Cancer 61, 89-95). 5T4 is expressed widely across many different tumor types, including lung, breast, head and neck, colorectal, bladder, ovarian, pancreatic, and many others (Stem, P. L. & Harrop, R. (2017) Cancer Immunol. Immunother. 66, 415-426). Favorable characteristics for targeting 5T4 with a radiolabeled targeting agent include its high rate of internalization, expression on the tumor periphery, and expression on cancer stem cells.

Several attempts have been made to develop therapeutics against tumors through 5T4 expression, including antibodies, vaccines, and cellular therapies. While an unlabeled 5T4-targeting antibody is not an effective therapeutic (Boghaert, et al. (2008) Int. J. Oncol. 32, 221-234), armed antibodies such as antibody drug-conjugates (ADC) with toxins have been developed and tested preclinically. Only an auristatin based ADC developed by Pfizer was tested clinically, with no objective responses reported and toxicity related to the auristatin conjugate observed (Shapiro, G. I. et al. (2017) Invest. New Drugs 35, 315-323).

Accordingly, radiotherapeutic agents of the presently disclosed invention include at least antibodies, peptides, and/or small molecules that target 5T4. Exemplary radiotherapeutics include ARCs targeted to 5T4, such as radiolabeled monoclonal antibodies against 5T4 (e.g., ²²⁵Ac-5T4). Exemplary antibodies against 5T4 include at least MED10641 developed by Medimmune/AstraZeneca; ALG.APV-527, developed by Aptevo Therapeutics/Alligator Bioscience; Tb535, developed by Biotecnol/Chiome Bioscience; H6-DM5 developed by Guangdong Zhongsheng Pharmaceuticals; and ZV0508 developed by Zova Biotherapeutics.

Radiotherapeutic Agents Targeting HER3

The human epidermal growth factor receptor 3 (ErbB3, also known as HER3) is a receptor protein tyrosine kinase belonging to the epidermal growth factor receptor (EGFR) subfamily of receptor protein tyrosine kinases. The transmembrane receptor HER3 consists of an extracellular ligand-binding domain having a dimerization domain therein, a transmembrane domain, an intracellular protein tyrosine kinase-like domain and a C-terminal phosphorylation domain. Unlike the other HER family members, the kinase domain of HER3 displays very low intrinsic kinase activity.

The ligands neuregulin 1 or neuregulin 2 bind to the extracellular domain of HER3 and activate receptor-mediated signaling pathway by promoting dimerization with other dimerization partners such as HER2. Heterodimerization results in activation and transphosphorylation of HER3's intracellular domain and is a means not only for signal diversification but also signal amplification. In addition, HER3 heterodimerization can occur in the absence of activating ligands and this is commonly termed ligand-independent HER3 activation. For example, when HER2 is expressed at high levels as a result of gene amplification (e.g. in breast, lung, ovarian or gastric cancer) spontaneous HER2/HER3 dimers can be formed. In this situation, the HER2/HER3 is considered the most active ErbB signaling dimer and is highly transforming.

Increased HER3 has been found in several types of cancer such as breast, lung, gastrointestinal and pancreatic cancers. Interestingly, a correlation between the expression of HER2/HER3 and the progression from a non-invasive to an invasive stage has been shown (Alimandi et al. (1995) Oncogene 10:1813-1821; DeFazio et al. (2000) Cancer 87:487-498).

Accordingly, radiotherapeutic agents of the presently disclosed invention include at least antibodies, peptides, and/or small molecules that target HER3. Exemplary radiotherapeutics include ARCs targeted to HER3, such as radiolabeled monoclonal antibodies against HER3 (e.g., ²²⁵Ac-HER3). Exemplary antibodies against HER3 include the monoclonal antibodies Patritumab, Seribantumab, Lumretuzumab, Elgemtumab, US-1402, AV-203, CDX-3379, and GSK2849330, the bispecific antibodies MM-111, MM-141/Istiratumab, MCLA-128, and MEHD7945A/Duligotumab, and the other anti-HER3 antibodies disclosed herein.

Exemplary anti-HER3 antibodies (also referred to as “HER3 antibodies” herein), such as anti-human HER3 antibodies, that that may be radiolabeled and embodied in and/or used in the various aspect of the presently disclosed invention include, without limitation, the following antibodies, and antibodies such as but not limited to immunoglobulins, such as but not limited to IgG, that (i) include the heavy chain variable region of the HER3 antibody or heavy chain, (ii) include 1, 2 or 3 of the heavy chain CDRs (e.g., by the Kabat definition) of the HER3 antibody or heavy chain or those recited, (iii) include the light chain variable region of the HER3 antibody or light chain, and/or (iv) include 1, 2 or 3 of the light chain CDRs (e.g., by the Kabat definition) of the HER3 antibody or light chain or those recited. It should also be understood that where a HER3 antibody heavy chain or HER3 antibody light chain is disclosed that includes an N-terminal leader sequence, also intended to be disclosed for embodiment in and use in the various aspects of the invention are corresponding heavy chains and corresponding light chains that lack the leader sequence.

An exemplary HER3 antibody that may be radiolabeled and embodied in and/or used in the presently disclosed invention may, for example, include a murine monoclonal antibody against HER3 including a heavy chain having the amino acid sequence as set forth in SEQ ID NO:9 or 11 and/or a light chain having the amino acid sequence as set forth in SEQ ID NO:10 or 12, or an antibody such as a humanized antibody derived from one or more of said sequences. An exemplary HER3 antibody that may be radiolabeled and embodied in and/or used in the presently disclosed invention may include or a heavy chain with an N-terminal region having the sequence set forth in SEQ ID NO:13 and/or a light chain with an N-terminal region having the sequence as set forth in SEQ ID NO: 14. A HER3 antibody that may be similarly embodied or used in various aspect of the invention may, for example, include the heavy chain variable region having the amino acid sequence as set forth in SEQ ID NO:7, and/or a light chain variable region having an amino acid sequence as set forth in SEQ ID NO:8; and/or a heavy chain including one or more of CDR1, CDR2 and CDR3 having the amino acid sequences respectively set forth in SEQ ID NOS:1-3, and/or a light chain with one or more of the CDR1, CD2 and CDR3 having the amino acid sequences respectively set forth in SEQ ID NOS:4-6. A HER3 antibody embodied in and/or used in any of the aspects of the invention may, for example, include any combination of the aforementioned light chain sequences and/or heavy chain sequences.

An exemplary HER3 antibody includes an immunoglobulin heavy chain variable region including a CDR-H1 including SEQ ID NO:15, a CDR-H2 including SEQ ID NO:16, and a CDR-H3 including SEQ ID NO:17, and/or an immunoglobulin light chain variable region including a CDR-L1 including SEQ ID NO:18, a CDR-L2 including SEQ ID NO:19, and a CDR-L3 including SEQ ID NO:20. An exemplary An exemplary HER3 antibody includes an immunoglobulin heavy chain variable region including SEQ ID NO:21 and/or an immunoglobulin light chain variable region including SEQ ID NO:22. An exemplary HER3 antibody includes an immunoglobulin heavy chain amino acid sequence of SEQ ID NO:23 and/or an immunoglobulin light chain amino acid sequence of SEQ ID NO:24.

An exemplary HER3 antibody includes an immunoglobulin heavy chain variable region including a CDR-H1 including SEQ ID NO:25, a CDR-H2 including SEQ ID NO:26, and a CDR-H3 including SEQ ID NO:27; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:31 and/or an immunoglobulin light chain variable region including SEQ ID NO:32. An exemplary HER3 antibody includes an immunoglobulin heavy chain amino acid sequence of SEQ ID NO:33 and/or an immunoglobulin light chain amino acid sequence of SEQ ID NO:34

An exemplary HER3 antibody includes an immunoglobulin heavy chain variable region including a CDR-H1 including SEQ ID NO:35, a CDR-H2 including SEQ ID NO:36, and a CDR-H3 including SEQ ID NO:37; and/or an immunoglobulin light chain variable region including a CDR-L1 including SEQ ID NO:38, a CDR-L2 including SEQ ID NO:39, and a CDR-L3 including SEQ ID NO:40. An exemplary HER3 antibody includes an immunoglobulin heavy chain variable region including SEQ ID NO:41, and/or an immunoglobulin light chain variable region SEQ ID NO:42. An exemplary HER3 antibody includes an immunoglobulin heavy chain amino acid sequence of SEQ ID NO:43 and an immunoglobulin light chain amino acid sequence of SEQ ID NO:44.

An exemplary HER3 antibody includes an immunoglobulin heavy chain variable region including a CDR-H1 including SEQ ID NO:45, a CDR-H2 including SEQ ID NO:46, and a CDR-H3 including SEQ ID NO:47; and/or an immunoglobulin light chain variable region including a CDR-L1 including SEQ ID NO:48, a CDR-L2 including SEQ ID NO:29, and a CDR-L3 including SEQ ID NO:49. An exemplary HER3 antibody includes an immunoglobulin heavy chain variable region including SEQ ID NO:50 and/or an immunoglobulin light chain variable region including SEQ ID NO:51. An exemplary HER3 antibody includes an immunoglobulin heavy chain amino acid sequence of SEQ ID NO:52 and/or an immunoglobulin light chain amino acid sequence of SEQ ID NO:53.

An exemplary HER3 antibody includes an immunoglobulin heavy chain variable region including a CDR-H1 including SEQ ID NO:54, a CDR-H2 including SEQ ID NO:55, and a CDR-H3 including SEQ ID NO:56; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:57 and/or an immunoglobulin light chain variable region including SEQ ID NO:58. An exemplary HER3 antibody includes an immunoglobulin heavy chain amino acid sequence of SEQ ID NO:59 and/or an immunoglobulin light chain amino acid sequence of SEQ ID NO: 60.

An exemplary HER3 antibody includes an immunoglobulin heavy chain variable region including a CDR-H1 including SEQ ID NO:61, a CDR-H2 including SEQ ID NO:62, and a CDR-H3 including SEQ ID NO:63; and/or an immunoglobulin light chain variable region including a CDR-L1 including SEQ ID NO:64, a CDR-L2 including SEQ ID NO:65, and a CDR-L3 including SEQ ID NO:66. An exemplary HER3 antibody includes an immunoglobulin heavy chain variable region including SEQ ID NO:67, and/or an immunoglobulin light chain variable region including SEQ ID NO:68. An exemplary HER3 antibody includes an immunoglobulin heavy chain amino acid sequence of SEQ ID NO:69 and an immunoglobulin light chain amino acid sequence of SEQ ID NO:70.

An exemplary HER3 antibody includes an immunoglobulin heavy chain variable region including a CDR-H1 including SEQ ID NO:71, a CDR-H2 including SEQ ID NO:72, and a CDR-H3 including SEQ ID NO:66; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:73, and/or an immunoglobulin light chain variable region including SEQ ID NO:74. An exemplary HER3 antibody includes an immunoglobulin heavy chain amino acid sequence of SEQ ID NO:75 and/or an immunoglobulin light chain amino acid sequence of SEQ ID NO:76.

An exemplary HER3 antibody includes an immunoglobulin heavy chain amino acid sequence of SEQ ID NO:77 and/or an immunoglobulin light chain amino acid sequence of SEQ ID NO:78.

An exemplary HER3 antibody includes an immunoglobulin light chain variable region including SEQ ID NOS:86, 87, 88, 89, 90 or 91 and/or a heavy chain variable region including SEQ ID NOS:79, 80, 81, 82, 83, 84 or 85.

An exemplary HER3 antibody includes an immunoglobulin heavy chain sequence including SEQ ID NO:92, 94, 95, 98 or 99 and/or an immunoglobulin light chain sequence including SEQ ID NO:93, 96, 97, 100 or 101.

Exemplary HER3 antibodies also include Barecetamab (ISU104) from Isu Abxis Co and any of the HER3 antibodies disclosed in U.S. Pat. No. 10,413,607.

Exemplary HER3 antibodies also include HMBD-001 (10D1F) from Hummingbird Bioscience Pte. and any of the HER3 antibodies disclosed in International Pub. Nos. WO 2019185164 and WO2019185878, U.S. Pat. No. 10,662,241; and U.S. Pub. Nos. 20190300624, 20210024651, and 20200308275.

Exemplary HER3 antibodies also include the HER2/HER3 bispecific antibody MCLA-128 (i.e., Zenocutuzumab) from Merus N.V.; and any of the HER3 antibodies, whether monospecific or multi-specific, disclosed in U.S. Pub. Nos. 20210206875, 20210155698, 20200102393, 20170058035, and 20170037145.

Exemplary HER3 antibodies also include the HER3 antibody Patritumab (U3-1287), an antibody including heavy chain sequence SEQ ID NO:106 and/or light chain sequence SEQ ID NO:7 which are reported chains of Patritumab, and any of the HER3 antibodies disclosed in U.S. Pat. Nos. 9,249,230 and 7,705,130 and International Pub. No. WO2007077028.

Exemplary HER3 antibodies also include the HER3 antibody MM-121 and any of the HER3 antibodies disclosed in U.S. Pat. No. 7,846,440 and International Pub. No. WO2008100624.Exemplary HER3 antibodies also include the EGFR/HER3 bispecific antibody DL1 and any of the HER3 antibodies, whether monospecific or multi-specific, disclosed in U.S. Pat. Nos. 9,327,035 and 8,597,652, U.S. Pub. No. 20140193414, and International Pub. No. WO2010108127.

Exemplary HER3 antibodies also include the HER2/HER3 bispecific antibody MM-111 and any of the HER3 antibodies, whether monospecific or multi-specific, disclosed in U.S. Pub. Nos. 20130183311 and 20090246206 and International Pub. Nos. WO2006091209 and WO2005117973.

According to certain aspects, the TER3 targeting agent includes an anti-HER3 antibody that binds to an epitope of cER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV2 and MP-RM-1 from MediPharma, Barecetamab (SU1d4) from esu Abxis Co., HNMID-001 (10D1F) from Hummingbird Bioscience Pte., REGN1400 from Regeneron Pharmaceuticals, and/or AV-203 from AVEO Oncology. According to certain aspects, the anti-HER3 antibody is selected from one or more of Patritumab, Seribantumab or an antibody including heavy chain sequence SEQ TD NO:108 and/or light chain sequence SEQ TD NO:109 which are reported for Seribantumab, Lumretuzumab or an antibody including heavy chain sequence SEQ ID NO:110 and/or light chain sequence SEQ TD NO:111 which are reported for Lumretuzumab, Elgemtumab or an antibody including heavy chain sequence SEQ ID NO: 112 and/or light chain sequence SEQ TD NO:113 which are reported for Elgemtumab, AV-203, CDX-3379, GSK2849330, EV20, MP-RM-1, ISU14, HMBD-001 (10D1F), and REGN1400. Exemplary antibodies along with exemplary treatment indications are also described in Table 2.

TABLE 2

| Company Name | Product | | Therapeutic | Exemplary |
|---|---|---|---|---|
| (Originator) | Name | Targets | Modality | Indications |
| Aveo Pharmaceuticals | CAN017, | HER3 | Antibody | Esophageal cancer, solid |
| Inc. | AV-203 | | | tumors |
| Celldex Therapeutics | CDX-3379, | HER3 | Antibody | Head and neck cancer, solid |
| Inc. | ktn3379 | | | tumors |
| Daiichi Sankyo Co. | patritumab | HER3 | Antibody | Non-small cell lung cancer |
| Ltd. | (AMG 888, | | | (NSCLC), breast cancer, |
| | U3-1287) | | | head and neck cancer |
| Daiichi Sankyo Co. | U3-1402 | HER3 | Antibody- | NSCLC, breast cancer, colon |
| Ltd. | | | drug | cancer |
| | | | conjugate | |
| GSK | GSK2849330 | HER3 | Antibody | Solid tumors |
| Hummingbird | HMBD-001 | HER3 | Antibody | Gastric cancer |
| Bioscience Pte. Ltd. | (10D1F) | | | |
| Isu Abxis Co. Ltd. | ISU104 | HER3 | Antibody | Cancer (unspecified) |
| MediPharma | MP-RM-1 | HER3 | Antibody | Solid tumors |
| MediPharma | EV20 | HER3 | Antibody | Solid tumors |
| Merrimack | Seribantumab | HER3 | Antibody | NSCLC, breast cancer, |
| Pharmaceuticals Inc. | (MM-121, | | | ovarian cancer |
| | SAR256212) | | | |
| Novartis AG | elgemtumab | HER3 | Antibody | Esophageal cancer, Breast |
| | (LJM716) | | | cancer, solid tumors |
| Regeneron | REGN1400 | HER3 | Antibody | Cancer (unspecified) |
| Pharmaceuticals Inc. | | | | |
| Roche | Lumretuzumab | HER3 | Antibody | Breast cancer, solid tumors |
| | (RG7116 or | | | |
| | RO5479599) | | | |

The sequence and structure of human HER3, human HER2, and human EGFR (HER1) are all known. An amino acid sequence of the human HER3 precursor protein (receptor tyrosine-protein kinase erbB-3 isoform 1 precursor NCBI Reference Sequence: NP_001973.2) is provided herein as SEQ ID NO:115. Those skilled in the art will readily appreciate that given known target protein amino acid sequences, various types of suitable antibodies and antibody mimetics specific for the extracellular domain of HER3, such as of human HER3, for use in the various aspects of the invention, may be produced using immunization and/or panning and/or antibody engineering techniques that are well established in the art.

A HER3 targeting agent that is radiolabeled for use in the various embodiments of the invention may, for example, include a HER3 binding peptide such as chelator-bearing HER3 binding peptide, such as a DOTA-bearing HER3 binding peptide, such as any of those disclosed in U.S. Pub. No. 20200121814.

Radiotherapeutic Agents Targeting TROP2

Tumor-associated calcium signal transducer 2, also known as Trop-2 and as epithelial glycoprotein-1 antigen (EGP-1), is a protein encoded by the human TACSTD2 gene which is overexpressed in carcinomas. Overexpression of TROP2 is associated with poor survival in human solid tumor patients. Cancers that may be targeted with a TROP2 targeting agent and treated with a radiolabeled TROP2 targeting agent according to the invention include but are not limited to carcinomas, squamous cell carcinomas, adenocarcinomas, non-small cell lung cancer (NSCLC), Small-cell lung cancer (SCLC), colorectal cancer, gastric adenocarcinoma, esophageal cancer, hepatocellular carcinoma, ovarian epithelial cancer, breast cancer, metastatic breast cancer, triple negative breast cancer (TNBC), prostate cancer, hormone-refractory prostate cancer, pancreatic ductal adenocarcinoma, head and neck cancers, renal cell cancer, urinary bladder neoplasms, cervical cancer, endometrial cancer, uterine cancer, follicular thyroid cancer, glioblastoma multiforme.

Exemplary TROP2 targeting agents that may be radiolabeled and used in conjunction with one or more CD47 blockades in the treatment of a proliferative disorder include the monoclonal antibodies Sacituzumab and Datopotamab, antibodies having one or both of the heavy chain and light chain of said antibodies, and antibodies having one or both of the heavy chain CDRs and the light chain CDRs of said antibodies, or TROP2-binding fragments of any of the aforementioned antibodies. Sacituzumab biosimilar is commercially available as Catalog No. A2175 from BioVision Incorporated (an Abcam company, Waltham, Mass., USA). Datopotamab biosimilar is commercially available as Catalog No. PX-TA1653 from ProteoGenix (Schiltigheim, France).

Exemplary TROP2 targeting agents that may be radiolabeled and used in conjunction with one or more CD47 blockade in the treatment of a proliferative disorder include a monoclonal antibody having a heavy chain SEQ ID NO:135 and/or a light chain SEQ ID NO:140 (reported as the heavy and light chains of Sacituzumab), or an antibody including one or both of the heavy chain variable region (SEQ ID NO:136) or the light chain variable region (SEQ ID NO:141) of said chains, or an antibody including 1, 2, or 3 of the heavy chain CDRs of said heavy chain (CDR H1-3: SEQ ID NOS:137-139 respectively) and/or 1, 2 or 3 of the light chain CDRs of said light chain (CDR L1-3: SEQ ID NOS:142-144 respectively), and any of the anti-human TROP antibodies disclosed in U.S. Pat. No. 7,238,785 (hRS7), U.S. Pat. No. 9,492,566, 10,195,517, or 11,116,846, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

Further exemplary TROP2 targeting agents that may be radiolabeled and used in conjunction with one or more CD47 blockade in the treatment of a proliferative disorder include a monoclonal antibody heavy chain SEQ ID NO:145 and/or a light chain SEQ ID NO:150 (reported as the heavy and light chains of Datopotamab), or an antibody including one or both of the variable region of said heavy chain (SEQ ID NO:146) and the variable region of said light chain (SEQ ID NO:151), or an antibody including 1, 2, or 3 of the heavy chain CDRs of said heavy chain (CDRs 1-3: SEQ ID NOS:147-149 respectively) and/or 1, 2 or 3 of the light chain CDRs of the said light chain (CDR H1-3: SEQ ID NOS:152-154 respectively), and any of the anti-human TROP antibodies disclosed in Int'l Pub. No. WO2015098099 or U.S. Pub. No. 20210238303, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

Radiotherapeutic Agents Targeting MUC1

Exemplary MUC1 targeting agents that may be radiolabeled and used in combination or conjunction with one or more CD47 blockades such as any of those disclosed herein for the treatment of a proliferative disorder such as a MUC1 expressing cancer, include hTAB004 (OncoTAb, Inc.) and any of the anti-MUC1 antibodies disclosed in any of U.S. Pub. No. 20200061216 and U.S. Pat. Nos. 8,518,405; 9,090,698; 9,217,038; 9,546,217; 10,017,580; 10,507,251 10,517,966; 10,919,973; 11,136,410; and 11,161,911. An exemplary radiolabeled MUC1 targeting agent that may be used in combination or conjunction with one or more CD47 blockades according to the invention is ⁹⁰Y IMMU-107 (hPAM4-Cide; Immunomedics, Inc.; Gilead Sciences, Inc.), or ¹⁷⁷Lu or ²²⁵Ac alternatively labeled versions thereof. Radiolabeled MUC1 targeting agents may be used in the treatment of MUC1 overexpressing cancers, such as MUC1 overexpressing solid tumors, such as pancreatic cancer, locally advanced or metastatic pancreatic cancer and breast cancer, such as metastatic breast cancer, tamoxifen-resistant breast cancer, HER2-negative breast cancer, and triple negative breast cancer (TNBC).

Radiotherapeutic Agents Targeting LYPD3 (C4.4A)

Exemplary LYPD3 (C4.4A) targeting agents that may be radiolabeled for use in combination or conjunction with one or more CD47 blockades according to the invention include, for example, BAY 1129980 (a/k/a Lupartumab amadotin; Bayer AG, Germany) an Auristatin-based anti-C4.4A (LYPD3) ADC or its antibody component Lupartumab, IgG₁ mAb GT-002 (Glycotope GmbH, Germany) and any of those disclosed in U.S. Pub. No. 20210309711, 20210238292, 20210164985, 20180031566, 20170158775, or 20150030618, 20120321619, Canadian Patent Application No. CA3124332A1, Taiwan Application No. TW202202521A, or Int'l Pub. No. WO2021260208, WO2007044756, WO2022042690, or WO2020138489. Such use may, for example, be for the treatment of a LYPD3-expressing hematological or solid tumor cancer in a mammal, such as carcinomas, primary and metastatic transitional cell carcinomas (TCCs), adenocarcinomas, lung cancer, lung adenocarcinoma, non-small cell lung cancer (NSCLC), hepatocellular carcinoma (HCC), breast cancer, endocrine therapy-resistant breast cancer (such as tamoxifen-resistant breast cancer), HER2-positive breast cancer, triple negative breast cancer (TNBC), esophageal cancer, renal cell carcinomas, colorectal cancer, cervical cancer, head and neck cancer, urothelial cancer, skin cancer, melanoma, and acute myelogenous leukemia (AML).

It should be understood that wherever in this disclosure specific antibodies, specific antibody heavy chains and specific antibody light chains are disclosed, against CD33, 5T4, DR5, HER2, HER3, TROP2 or against any target, also intended to be disclosed for embodiment in or use in the various aspects of the invention are antibodies, such as but not limited to immunoglobulins, such as but not limited to IgG, that (i) include the heavy chain variable region of the disclosed antibody or heavy chain, (ii) include 1, 2 or 3 of the heavy chain CDRs (e.g., by Kabat definition) of the disclosed antibody or heavy chain, (iii) include the light chain variable region of the disclosed antibody or light chain, and/or (iv) include 1, 2 or 3 of the light chain CDRs (e.g., by Kabat definition) of the disclosed antibody or light chain. It should also be understood that wherever in this disclosure an antibody heavy chain or an antibody light chain is disclosed that includes an N-terminal leader sequence, also intended to be disclosed for embodiment in and use in the various aspects of the invention are corresponding heavy chains and corresponding light chains that lack the leader sequence.

Radiotherapeutic Agents Targeting PSMA

In one aspect of the invention the radiolabeled targeting agent used in combination or conjunction with a one or more CD47 blocked may be a radiolabeled PSMA-targeting agent such as a radiolabeled anti-PSMA monoclonal antibody such as J591 labeled for example with ¹⁷⁷Lu or ²²⁵Ac or Rosopatamab labeled for example with ¹⁷⁷Lu or ²²⁵Ac, or a radiolabeled PSMA-binding small molecule such as PSMA-617 labeled for example with ¹⁷⁷Lu or ²²⁵Ac, PSMA I&T labeled for example with ¹⁷⁷Lu or ²²⁵Ac, FrhPSMA-7 labeled for example with ¹⁷⁷Lu, 64/67Cu-SAR-bisPSMA (Clarity Pharmaceuticals), CONV 01-α (Convergent Therapeutics, Inc.) labeled for example with ²²⁵Ac, ¹⁷⁷Lu-PSMA I&T-β+²²⁵Ac-CONV01-α combination (Convergent Therapeutics, Inc.), ¹³¹I-1095 (Lantheus Holdings/Progenics Pharmaceuticals, Inc.), ¹³¹I PSMA-PK-Rx (Noria Therapeutics, Inc.; Bayer), or PSMA-R2 labeled for example with ¹⁷⁷Lu, CTT1403 (Cancer Targeted Technology LLC) labeled for example with ¹⁷⁷Lu, PNT2002/Lu-177-PSMA-I&T (Point Biopharma Global Inc.), PNT2002/Lu-177-PSMA-I&T+²²⁵Ac-J591, TLX591 (¹⁷⁷Lu-Rosopatamab; Telix Pharmaceuticals Ltd.), TLX-591-CHO (Telix Pharmaceuticals Ltd.), and ¹⁷⁷Lu-EB-PSMA-617 (Sinotau Radiopharmaceutical). Such agents may, for example, be used in the treatment of prostate cancer, such as metastatic prostate cancer, castration-resistant prostate cancer (CRPC), metastatic CRPC (mCRPC), and/or hormone therapy resistant prostate cancer (anti-androgen therapy resistant prostate cancer) in combination with or in conjunction with one or more CD47 blockades according to the invention. Any of the agents that include DOTA or a DOTA derivative as a chelator may alternatively be labeled with any therapeutically active radionuclide that can be chelated by DOTA, such as ²²⁵Ac, ¹⁷⁷Lu and ⁹⁰Y.

Other Radiolabeled Cancer Targeting Agents

The radiolabeled cancer targeting agent used in combination or conjunction with one or more CD47 blockades, such as any of those disclosed herein, may for example also be any of the following radiolabeled targeting agents:

a radiolabeled FAP targeting agent such as ¹⁷⁷Lu-FAP-2286 (Clovis Oncology, Inc.) to treat, for example, solid tumors or any of the cancers disclosed herein;

a radiolabeled CCK2R targeting agents such as DEBIO 1124/¹⁷⁷Lu-DOTA-PP-F11N (Debiopharm International SA) to treat, for example, advanced, unresectable pulmonary extrapulmonary small cell carcinoma, and thyroid cancer such as metastatic thyroid cancer, or any of the cancers disclosed herein;

a radiolabeled CDH3 (cadherin-3, P-cadherin) targeting agent such as ⁹⁰Y labeled FF-21101 (FujiFilm Holdings Corporation/FujiFilm Toyama Chemical) to treat, for example, solid tumors such as epithelial ovarian peritoneal or fallopian tube carcinoma, TNBC, head and neck squamous cell carcinoma (HNSCC), cholangiocarcinoma, pancreatic, colorectal cancer, or any of the cancers disclosed herein;

a radiolabeled IGF-R1 targeting agent such as ²²⁵Ac FPI-1434 (Fusion Pharmaceuticals, Inc.) to treat, for example, solid tumors expressing IGF-R1, or any of the cancers disclosed herein;

a radiolabeled CEACAM5 targeting agent such as ⁹⁰Y-hMN14 and ⁹⁰Y TF2 (Immunomedics, Inc.; Gilead Sciences Inc.) to treat, for example, solid tumors such as colon cancer, colorectal cancer, pancreatic cancer, breast cancer such as HER-negative breast cancer, and thyroid cancer such medullary thyroid carcinoma, or any of the cancers disclosed herein;

a radiolabeled CD22 targeting agent such as IMMU-102 (⁹⁰Y-epratuzumab) (Immunomedics, Inc.; Gilead Sciences Inc.) to treat, for example, hematological malignancies such as CD22-positive acute lymphoblastic leukemia, non-Hodgkin lymphoma (NHL), stage III/IV DLBCL, follicular lymphoma, or any of the cancers disclosed herein;

a radiolabeled SSTR2 targeting agent such as Lutathera™ (lutetium Lu 177 dotatate; 177Lu-DOTA0-Tyr3-Octreotate; Novartis), Lutathera™ (lutetium Lu 177 dotatate)+⁹⁰Y-DOTATATE combination (Novartis), ¹⁷⁷LU-OPS201 (Ipsen Pharmaceuticals) the combination¹⁷⁷LU-OPS201/¹⁷⁷Lu-IPN01072 (Ipsen Pharmaceuticals), EBTATE (¹⁷⁷Lu-DOTA-EB-TATE; Molecular Targeting Technologies, Inc.), ORM2110 (AlphaMedix™; Orano Med), and PNT2003 labeled for example with ¹⁷⁷Lu (Point Biopharma Global Inc.), for the treatment of SSTR2 expressing cancers such as solid tumors, for example, neuroendocrine tumors, small cell lung cancer, breast cancer, prostate cancer such as metastatic prostate cancer, such as metastatic castration-resistant prostate cancer, neuroendocrine tumors, gastroenteropancreatico neuroendocrine tumors (GEP-NET), as well as such as locally advanced or metastatic forms thereof, or any of the cancers disclosed herein;

a radiolabeled SSTR2 and SSTR5 targeting agent such as Solucin™ (¹⁷⁷Lu-Edotreotide; Isotopen Technologien München AG (ITM)) to treat, for example, neuroendocrine tumors, or any of the cancers disclosed herein;

a radiolabeled Neurotensin receptor type 1 (NTSR1) targeting agent such as ¹⁷⁷Lu-IPN01087/¹⁷⁷Lu-3BP-227 or (Ipsen Pharmaceuticals) to treat, for example, solid tumors expressing NTSR1 such as pancreatic ductal adenocarcinoma, colorectal cancer, gastric cancer, squamous cell carcinoma of the head and neck, bone cancer, advanced cancer, recurrent disease, metastatic tumors, or any of the cancers disclosed herein;

a radiolabeled human Kallikrein-2 (hK2) targeting agent such as JNJ-69086420 (Janssen/Janssen Pharmaceutica NV) labeled for example with m¹⁵Ac, to treat, for example, prostate cancer such as locally advance or metastatic prostate cancer, or any of the cancers disclosed herein;

a radiolabeled NET (via norepinephrine transporter) targeting agent such as 131I-MIBG (Jubilant Radioharma) to treat, for example, neuroblastoma such as relapsed/refractory neuroblastoma, or any of the cancers disclosed herein;

a radiolabeled neuroepinephrine transporter targeting agents such as Azedra™ (iobenguane ¹³¹I; Lantheus Holdings/Progenics Pharmaceuticals, Inc.) to treat, for example, glioma, paraglioma, malignant pheochromocytoma/paraganglioma, and malignant relapsed/refractory pheochromocytoma/paraganglioma, or any of the cancers disclosed herein;

a radiolabeled Integrin αVβ6 targeting agent such as DOTA-ABM-5G, αVβ6 Binding Peptide (ABP; Luminance Biosciences, Inc.) labeled for example with ¹⁷⁷Lu, ²²⁵Ac or ⁹⁰Y, to treat, for example, solid tumors such as pancreatic cancer, or any of the cancers disclosed herein;

a radiolabeled CD37 targeting agent such as Betalutin™ (¹⁷⁷Lu-lilotomab satetraxetan; Nordic Nanovector ASA) to treat, for example, hematological malignancies such as lymphomas, such as follicular lymphoma or non-Hodgkin lymphoma (NHL) such as relapsed and/or refractory forms thereof, or any of the cancers disclosed herein;

a radiolabeled GRPR targeting agent such as ¹⁷⁷Lu-NeoB (Novartis) and ²¹²Pb-DOTAM-GRPR1 (Orano Med) to treat GRPR-expressing cancers, for example, prostate cancer, such as advanced prostrate cancer, locally advanced prostate cancer, metastatic prostate cancer, and castration-resistant prostate cancer, or any of the cancers disclosed herein;

a radiolabeled CXCR4 targeting agents such as PentixaTher™ (PentixaPharm GmbH) labeled with ¹⁷⁷Lu, ⁹⁰Y or ²²⁵Ac to treat, for example, lymphoproliferative or myeloid malignancies, including relapsed and/or refractory forms thereof, or any of the cancers disclosed herein;

a radiolabeled Tenascin-C targeting agent such as ¹³¹I-F16SIP (Philogen S.p.A.) to treat, for example, solid tumors or hematological malignancies such as any of those disclosed herein;

a radiolabeled Fibronectin extradomain B (EBD) targeting agent such as 131I-L19SIP (Philogen S.p.A.)) to treat, for example, solid tumors such as solid tumor brain metastases and non-small cell lung cancer (NSCLC), or any of the cancers disclosed herein;

a radiolabeled LAT-1 targeting agent such as 4-¹³¹Iodo-L-phenylalanine (Telix Pharmaceuticals Ltd.) to treat, for example, glioblastoma such as recurrent glioblastoma, or any of the cancers disclosed herein;

a radiolabeled Carbonic Anhydrase IX (CAIX) targeting agent such as radiolabeled Girentuxumab (cG250) such as DOTA conjugated Girentuxumab (cG250) labeled for example with ¹⁷⁷Lu (such as TLX250; Telix Pharmaceuticals Ltd.), ²²⁵Ac or ⁹⁰Y, to treat, for example, renal cell carcinoma, such as ccRCC, or any of the cancers disclosed herein;

a radiolabeled CD66 targeting agent such as ⁹⁰Y-besilesomab (⁹⁰Y-anti-CD66-MTR; Telix Pharmaceuticals Ltd.) to treat, for example, leukemias, myelomas and lymphomas, such as any of those disclosed herein including pediatric and adult forms, or any of the cancers disclosed herein;

a radiolabeled B7-H3 targeting agents such as radiolabeled omburtumab, such ¹³¹I-8H9 (131I-omburtumab; Y-mAbs Therapeutics, Inc.) and ¹⁷⁷Lu-omburtamab (Y-mAbs Therapeutics, Inc.) to treat, for example, gliomas such as non-progressive diffuse pontine gliomas, such as non-progressive diffuse pontine gliomas previously treated with external beam radiation therapy, brain tumors, central nervous system tumors, neuroblastomas, sarcomas, leptomeningeal metastasis from solid tumors, and medulloblastoma, including in pediatric and adult forms, or any of the cancers disclosed herein;

a radiolabeled NKG2D ligand targeting agent such as a radiolabeled recombinant human NKG2D Fc chimeric protein, for example, Catalog No. 1299-NK from Biotechne (R&D Systems, Inc., Minneapolis, Minn., USA) which includes Phe78-Val216 of Human NKG2D (Accession #P26718) or a radiolabeled recombinant human NKG2D Fc chimeric protein including SEQ ID NO:155 plus/minus an amino-terminal histidine tag such as (His)₆, or a radiolabeled antibody or antigen-binding fragment thereof against an NKG2D ligand such as MICA, MICB, RAET1E/ULBP4, RAET1G/ULBP5, RAET1H/ULBP2, RAET1/ULBP1, RAET1L/ULBP6, or RAET1N/ULBP3—to treat, for example solid tumors or hematological malignancies expressing one or more NKG2D ligands, or any of the cancers disclosed herein;

a radiolabeled GD2 targeting agent such as GD2-SADA:¹⁷⁷Lu-DOTA (Y-mAbs Therapeutics, Inc.) to treat, for example, SCLC, melanoma, sarcoma or any of the cancers disclosed herein;

a radiolabeled Folate receptor alpha (FOLR1) targeting agent such as a radiolabeled anti-FOLR1 antibody such as radiolabeled Mirvetuximab or Farletuzumab, to treat, for example, solid cancers such as ovarian cancer, lung cancer, NSCLC, breast cancer, TNBC, brain cancer, glioblastoma, colorectal cancer or any of the cancers disclosed herein;

a radiolabeled Nectin-4 targeting agent, such as a radiolabeled anti-Nectin-4 monoclonal antibody such as radiolabeled Enfortumab or radiolabeled forms of any of the anti-Nectin-4 antibodies or targeting agents disclosed in U.S. Pub. No. 20210130459, U.S. Pub. No. 20200231670, U.S. Pat. No. 10,675,357, or Int'l Pub. No. WO2022051591, to treat, for example, solid tumors such as urothelial carcinoma, bladder carcinoma, breast cancer, TNBC, lung cancer, NSCLC, colorectal cancer, pancreatic cancer, endometrial cancer, ovarian cancer or any of the cancers disclosed herein;

a radiolabeled CUB-domain containing protein 1 (CDCP1) targeting agent such as a radiolabeled monoclonal antibody such as radiolabeled forms of any of the CDCP1 targeting agents and antibodies disclosed in U.S. Pub. No. 20210179729, U.S. Pub. No. 20200181281, U.S. Pub. No. 20090196873, U.S. Pat. Nos. 8,883,159, 9,346,886, or Int'l Pub No. WO2021087575, to treat, for example, solid cancers such as breast cancer, TNBC, lung cancer, colorectal cancer, ovarian cancer, kidney cancer, liver cancer, HCC, pancreatic cancer, skin cancer, melanoma, or a hematological malignancy such as acute myeloid leukemia, or any of the cancers disclosed herein;

a radiolabeled Glypican-3 (GPC3) targeting agent such as a radiolabeled anti-GPC3 mAb such as the radiolabeled humanized IgG₁ mAb GC33 (a/k/a Codrituzumab; commercially available as Catalog No. TAB-H14 from Creative Biolabs), such as ²²⁵Ac-Macropa-GC33 (Bell et al., Glypican-3-Targeted Alpha Particle Therapy for Hepatocellular Carcinoma. Molecules. 2020 Dec. 22; 26(1):4.) or a radiolabeled form of any of the anti-GPC3 antibodies or other targeting agents disclosed in U.S. Pat. Nos. 10,118,959, 10,093,746, 10,752,697, 9,932,406, 9,217,033, 8,263,077, 7,871,613, 7,867,734, U.S. Pub. No. 20190046659, U.S. Pub. No. 20180243451, U.S. Pub. No. 20170369561, or U.S. Pub. No. 20150315278, to treat GPC3-expressing cancers such as hepatocellular carcinoma, ovarian clear cell carcinoma, melanoma, NSCLC, squamous cell carcinoma of the lung, hepatoblastoma, nephroblastoma (Wilms tumor), yolk sac tumor, gastric carcinoma, colorectal carcinoma, head and neck cancer, and breast cancer.

a radiolabeled urokinase plasminogen activator receptor (uPAR) targeting agent, such as a radiolabeled monoclonal antibody such as radiolabeled MINPR-101 (huATN-658) such as MNPR-101-PTCA-Ac225 (Monopar Therapeutics, Inc., Wilmette, Ill., USA) or radiolabeled forms of any of the anti-uPAR antibodies or targeting agents disclosed in U.S. Pat. No. 9,029,509, U.S. Pub. No. 20080199476, U.S. Pub. No. 20040204348 or Int'l Pub. No. WO2021257552, to treat, for example, solid cancers or hematological malignancies such as any of those disclosed herein; and/or

a radiolabeled LewisY antigen (LeY) targeting agent such as a radiolabeled anti-LeY monoclonal antibody such as radiolabeled forms of 3S1931 and/or of a humanized version thereof such as Hu3S1933, or of any of monoclonal antibodies B34, BR55-2, BR55/BR96, and IGN 133, or antigen binding fragments of any of the preceding antibodies, to treat, for example, solid tumors such as squamous cell lung carcinoma, lung adenocarcinoma, ovarian carcinoma, or colorectal adenocarcinoma or any of the cancers disclosed herein.

In still further embodiments of the invention, a radiolabeled targeting agent used in combination or conjunction with one or more CD47 blockades for the treatment of a cancer or proliferative disorder such as any of those disclosed herein in a mammal, such as a human, includes a phospholipid-based cancer targeting agent. In certain embodiments, the phospholipid-based cancer targeting agent includes any of the radioactive phospholipid metal chelates disclosed in U.S. Pub. No. 20200291049, incorporated by reference herein, such as but not limited to

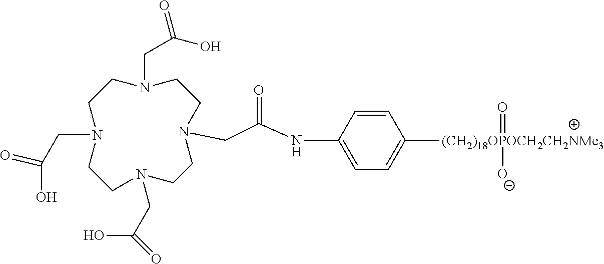

(a/k/a NM600) or a pharmaceutically acceptable salt thereof, chelated with a radionuclide, such as ²²⁵Ac, ¹⁷Lu, or ⁹⁰Y.

In certain aspects, the lipid based radiolabeled targeting agent used in conjunction with one or more CD47 blockades includes any of the radiolabeled phospholipid compounds disclosed in U.S. Pub. No. 20140030187 or U.S. Pat. No. 6,417,384, each incorporated by reference herein, such as but not limited to

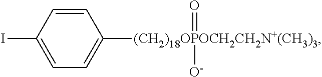

i.e., 18-(p-iodophenyl)octadecyl phosphocholine, wherein iodine is ¹³¹I (a/k/a NM404 I-131, and CLR 131), or a pharmaceutically acceptable salt thereof. In certain aspects, the phospholipid-based radiolabeled targeting agent used in conjunction with one or more CD47 blockades includes any of the phospholipid drug conjugate compounds disclosed in U.S. Pat. No. 9,480,754, incorporated by reference herein.

Multi-Specific Targeting Aspects

While an exemplary radiotherapeutic disclosed herein may include an antibody radioconjugate (ARC) against a single antigen, such as CD33, DR5, 5T4, HER2, HER3, or TROP2 multi-specific antibodies are also within the scope of the present invention. Thus, according to certain aspects, the radiotherapeutic may include a multi-specific targeting agent, such as a multi-specific antibody, that recognizes a first epitope of an antigen (such as CD33, DR5, 5T4, HER2, HER3, TROP2 or any of the cancer-associated antigen targets disclosed herein) and a second epitope of the same antigen, or recognizes an epitope of a first antigen and an epitope of one or more different antigens selected, for example, from any of the cancer-associated antigens disclosed herein. Thus, in one aspect, an ARC may include a multi-specific antibody), such as a bispecific antibody, that includes at least a first target recognition component which specifically binds to an epitope of a first antigen (such as CD33, DR5, 5T4, HER2, HER3, TROP2 or any of the cancer-associated antigen targets disclosed herein) and a second target recognition component which specifically binds to an epitope of an antigen other than the first antigen, such as any of the cancer-associated antigens disclosed herein.

The invention also provides compositions and methods for treatment of a proliferative disorder such as any of those disclosed herein that include or utilize at least two discrete radiolabeled targeting agents wherein the two targeting agents have specificity against different cancer-associated antigens and/or different cancer/tumor targeting mechanisms, and which targeting agents may for example, be any of those disclosed herein and/or may be directed against any of the targets disclosed herein.

In the various aspects of the invention, the cancer-associated antigen or antigens for which a radiolabeled targeting agent (such as an ARC) has specificity may, for example, include one or more of the following: CD33, DR5, 5T4, HER2 (ERBB2; Her2/neu), HER3, TROP2, mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), LYPD3 (C4.4A), Nectin-4, urokinase plasminogen activator receptor (uPAR), Folate receptor alpha (FOLR1), CUB-domain containing protein 1 (CDCP1), Glypican-3 (GPC3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H3, KIT, IL-13Ra2, interleukin-11 receptor a (IL-1 1Ra), PSCA, PRSS21, VEGFR2, LewisY, CD24, platelet-derived growth factor receptor-beta (PDGFR-beta), SSEA-4, CD20, Folate receptor alpha (FRa), MUC1, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

The first target recognition component may, for example, include one of: a first full-length heavy chain and a first full-length light chain, a first Fab fragment, or a first single-chain variable fragment (scFvs). The first target recognition component may, for example, be derived from any of the monoclonal antibodies listed herein that are directed against CD33, DR5, 5T4, HER2, or HER3. The second target recognition component may, for example, include one of: a second full-length heavy chain and a second full-length light chain, a second Fab fragment, or a second single-chain variable fragment (scFvs). Moreover, the second target recognition component may be derived from any of the additional different antigens listed above.

Alternatively, the presently disclosed invention contemplates methods which include administration of a first ARC against at least one first antigen (i.e., CD33, DR5, 5T4, HER2, or HER3), and administration of a second ARC, wherein the second ARC is against a different epitope of the first antigen, or is against an epitope of a different antigen, such as an antigen selected from the list presented above, or another of the antigens against CD33, DR5, 5T4, HER2, or HER3 not targeted by the first ARC.

According to certain aspects, the effective amount of the radiotherapeutic, such as any of the ARCs disclosed herein, includes a maximum tolerated dose (MTD).

According to certain aspects, when more than one ARC or other cancer-targeting radiotherapeutic is administered, the agents may be administered at the same time. As such, according to certain aspects of the present invention, the agents may, for example, be provided in a single composition. Alternatively, the two radiotherapeutics may be administered sequentially. As such, a first ARC or other cancer-targeting radiotherapeutic may be administered before a second ARC or other cancer-targeting radiotherapeutic, after the second ARC or other cancer-targeting radiotherapeutic, or both before and after the second ARC or other cancer-targeting radiotherapeutic. Moreover, the second ARC or other cancer-targeting radiotherapeutic may be administered before the first ARC or other cancer-targeting radiotherapeutic, after the first ARC or other cancer-targeting radiotherapeutic, or both before and after the first ARC or other cancer-targeting radiotherapeutic.

According to certain aspects, the ARC or other cancer-targeting radiotherapeutic may be administered according to a dosing schedule selected from the group consisting of one every 7, 10, 12, 14, 20, 24, 28, 35, and 42 days throughout a treatment period, wherein the treatment period includes at least two doses.

According to certain aspects, the ARC or other cancer-targeting radiotherapeutic may be administered according to a dose schedule that includes 2 doses, such as on days 1 and 5, 6, 7, 8, 9, or 10 of a treatment period, or days 1 and 8 of a treatment period.

Administration of the ARCs of the present invention, in addition to other therapeutic agents, may be provided in several ways depending upon whether local or systemic treatment is desired and upon the area to be treated. Administration may be intratracheal, intranasal, epidermal and transdermal, oral or parenteral. Parenteral administration includes intravenous, intra-arterial, subcutaneous, intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may be administered as a single treatment or in a series of treatments that continue as needed and for a duration of time that causes one or more symptoms of the cancer to be reduced or ameliorated, or that achieves another desired effect.

The dose(s) may vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

The radiotherapeutics disclosed herein, such as any of the ARCs, may be administered simultaneously or sequentially with the one or more additional therapeutic agents. Moreover, when more than one additional therapeutic agent is included, the additional therapeutic agents may be administered simultaneously or sequentially with each other and/or with the radiotherapeutic.

Radiolabeling the Cancer Targeting Agent

The radiotherapeutic may be labeled with a radioisotope such as an alpha emitter (e.g., ²²⁵Ac) through conjugation of a chelator molecule, and chelation of the radioisotope. According to certain aspects, the radiotherapeutic may be an antibody against that is deglycosylated in the constant region, such as at asparagine-297 (Asn-297, N297; kabat number) in the heavy chain CH2 domain, for the purpose of uncovering a unique conjugation site, glutamine (i.e., Gln-295, Q295) so that it is available for conjugation with bifunctional chelator molecules.

According to certain aspects, the radiotherapeutic may be an antibody that may have reduced disulfide bonds such as by using reducing agents, which may then be converted to dehydroalanine for the purpose of conjugating with a bifunctional chelator molecule.

According to certain aspects, the radiotherapeutic may be an antibody for which the disulfide bonds have been reduced using reducing agents, which is then conjugated via aryl bridges with a bifunctional chelator molecule. For example, according to certain aspects a linker molecule such as 3,5-bis(bromomethyl)benzene may be used to bridge the free sulfhydryl groups on the antibody.

According to certain aspects, the radiotherapeutic may be an antibody that may have certain specific existing amino acids replaced with cysteine(s) that then can be used for site-specific labeling.

According to certain aspects, the radiotherapeutic may be radiolabeled through site-specific conjugation of suitable bifunctional chelators. Exemplary chelator molecules that may be used include p-SCN-Bn-DOTA, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂-(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP.

The chelator molecules may, for example, be attached to a targeting agent through a linker molecule. Exemplary linker molecules include:

—CH₂(C₆H₄)NH₂ or —CH₂(C₆H₄)NH—X—Y,

wherein X is

—R₂—CH₂CH₂O(CH₂CH₂O)ₙCH₂CH₂—,

—R₂—CH₂CH₂NHC(O)CH₂CH₂O(CH₂CH₂O)ₙCH₂CH₂—,

—R₂—(CH₂)ₙCH₂—,

—R₂—CH₂CH₂NHC(O)(CH₂)ₙCH₂—,

—R₂—CH(C(O)R₃)CH₂—, wherein R₃ is —OH or a short peptide (1-20 amino acids),

—R₂—CH₂CH₂O(CH₂CH₂O)ₙCH₂C(O)O—, or

—R₂—CH₂CH₂NHC(O)CH₂CH₂O(CH₂CH₂O)ₙCH₂CC(O)O—,

wherein n is 1-20, and

R₂ is —C(O)— or —C(S)NH—; and

Y is —NH₂ or —SR₄—, wherein R₄ is —H or —CH₂-3,5-bis(bromomethyl)benzene.

Targeting agents, such as protein targeting agents, for example antibodies and antigen-binding antibody fragments, and peptide targeting agents may, for example, be conjugated with a chelator for radiolabeling the targeting agent via chelation of a radionuclide. Such protein or peptide targeting agents, for example, that include lysine(s), may conveniently be conjugated to a DOTA chelating moiety using the bifunctional agent S-2-(4-Isothiocyanatobenzyl)-1,4,7,10-tetraazacyclododecane tetraacetic acid a/k/a/ “p-SCN-Bn-DOTA” (Catalog #B205; Macrocyclics, Inc., Plano, Tex., USA). p-SCN-Bn-DOTA may be synthesized by a multi-step organic synthesis fully described in U.S. Pat. No. 4,923,985. Chelation of a radionuclide by the DOTA moiety may be performed prior to chemical conjugation of the antibody with p-SCN-Bn-DOTA and/or after said conjugation.

Methods for labeling a chelator-conjugated targeting agent with an exemplary radionuclide are described in in Example 1.

CD47 Blockades

As used herein, the term “CD47 blockade” refers to any agent that reduces the binding of CD47 (e.g., on a target cell) to SIRPα (e.g., on a phagocytic cell). Non-limiting examples of suitable anti-CD47 reagents include SIRPα reagents, including without limitation SIRPα polypeptides, anti-SIRPα antibodies, soluble CD47 polypeptides, and anti-CD47 antibodies or antibody fragments. According to certain aspects, a suitable anti-CD47 agent (e.g. an anti-CD47 antibody, a SIRPα reagent, etc.) specifically binds CD47 to reduce the binding of CD47 to SIRPα. An agent for use in the methods of the invention will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

According to certain aspects, a SIRPα reagent may include the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. For example, the CD47 blocking agent may be any of those disclosed in U.S. Pat. No. 9,969,789 including but not limited to the SIRPα-IgG Fc fusion proteins disclosed therein, such as TTI-621 and TTI-622.

According to certain aspects, an anti-CD47 agent includes an antibody that specifically binds CD47 (i.e., an anti-CD47 antibody) and reduces the interaction between CD47 on one cell (e.g., an infected or malignant cell) and SIRPα on another cell (e.g., a phagocytic cell). Non-limiting examples of suitable antibodies include clones B6H12, 5F9, 8B6, and C3 (for example as described in International Pub. No. WO 2011/143624). Suitable anti-CD47 antibodies include fully human, humanized or chimeric versions of such antibodies.

Exemplary human or humanized antibodies especially useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK 117 from Akesobio Australia Pty; IMC-002 from Innovent Biologics; ZL-1201 from Zia Lab; SHR-1603 from Jiangsu HengRui Medicine Co.; and SRF231 from Surface Oncology. Bispecific monoclonal antibodies are also available, such as IBI-322, targeting both CD47 and PD-L1 from Innovent Biologics.

AO-176, in addition to inducing tumor phagocytosis through blocking the CD47-SIRPα interaction, has been found to preferentially bind tumor cells versus normal cells (particularly RBCs where binding is negligible) and directly kills tumor versus normal cells.

Antibodies against SIRPα may also be used as CD47 blockades. Without limitation, anti-SIRPα antibodies (also referred to as SIRPα antibodies herein) that may be used in or embodied in any of the aspects of the invention include but are not limited to the following anti-SIRPα antibodies, antibodies that include one or both of the heavy chain and light chain variable regions of the following anti-SIRPα antibodies, antibodies that include one or both of the heavy chain and the light chain CDRs of any of the following anti-SIRPα antibodies, and antigen-binding fragments of any of said anti-SIRPα antibodies:
(1) ADU-1805 (Sairopa B.V.; Aduro) and any of the SIRPα antibodies disclosed in Intl. Pub. No. WO2018190719 or U.S. Pat. No. 10,851,164;
(2) AL008 (Alector LLC) and any of the SIRPα antibodies disclosed in Intl. Pub. No. WO2018107058, U.S. Pub. No. 20190275150, or U.S. Pub. No. 20210179728;
(3) AL008 (Apexigen, Inc.) and any of the SIRPα antibodies disclosed in Intl. Pub. No. WO2021174127 or U.S. App. No. 63/108,547;
(4) SIRP-1 and SIRP-2 (Arch Oncology, Inc.) and any of the SIRPα antibodies disclosed in Intl. Pub. No. WO2021222746, U.S. App. No. 63/107,200 or U.S. Pub. No. 20200297842;
(5) OSE-172 (a/k/a BI 765063; Boehringer Ingelheim) and any of the SIRPα antibodies disclosed in Intl. Pub. No. WO2017178653 or U.S. Pub. No. 20190127477;
(6) CC-95251 (Bristol Myers Squibb; Celgene) and any of the SIRPα antibodies disclosed in Intl. Pub. No. WO2020068752 or U.S. Pub. No. 20200102387;
(7) ES004 (Elpiscience Biopharma) and any of the SIRPα antibodies disclosed in Intl. Pub. No. WO2021032078 or U.S. Pub. No. 20210347908;
(8) FSI-189 (Gilead Sciences, Inc.; Forty Seven) and any of the SIRPα antibodies disclosed in Intl. Pub. No. WO2019023347, U.S. Pat. No. 10,961,318 or U.S. Pub. No. 20210171654;
(9) BYON4228 (Byondis B.V.; Synthon) and any of the SIRPα antibodies disclosed in Intl. Pub. No. WO2018210793, Intl. Pub. No. WO2018210795, or U.S. Pub. No. 20210070874;
(10) any of the SIRPα antibodies disclosed in Intl. Pub. No. WO2018057669, U.S. Pat. No. 11,242,404 or U.S. Pub. No. 20220002434 (Alexo Therapeutics Inc., now ALX Oncology Inc.);
(11) any of the SIRPα antibodies disclosed in Intl. Pub. No. WO2015138600, U.S. Pat. No. 10,781,256 or 10,081,680 (Leland Stanford Junior University);
(12) BR105 (Bioray Pharma); or
(13) BSI-050 (Biosion, Inc.).

Other CD47 blockades that may be employed include any of those disclosed in U.S. Pat. No. 9,969,789 including without limitation the SIRPα-IgG Fc fusion proteins TTI-621 and TTI-622 (Trillium Therapeutics, Inc.), both of which preferentially bind CD47 on tumor cells while also engaging activating Fc receptors. A SIRPα-IgG Fc fusion protein including the amino acid sequence SEQ ID NO: 116, SEQ ID NO: 117, or SEQ ID NO: 118 may, for example, be used. Still other SIRPα Fc domain fusions proteins that may be used include ALX148 from Alx Oncology or any of those disclosed in Int'l Pub. No WO2017027422 or U.S. Pat. No. 10,696,730.

The CD47 blockade may alternatively, or additionally, include agents that modulate the expression of CD47 and/or SIRPα˜, such as phosphorodiamidate morpholino oligomers (PMO) that block translation of CD47 such as MBT-001 (PMO, morpholino, Sequence: 5′-CGTCACAGGCAGGACCCACTGCCCA-3′) [SEQ TD NO: 114]) or any of the PMO oligomer CD47 inhibitors disclosed in any of U.S. Pat. Nos. 8,557,788, 8,236,313, 10,370,439 and Int'l Pub. No. WO2008060785.

Small molecule inhibitors of the CD47-SIRPα axis may also be used, such as RRx-001 (1-bromoacetyl-3,3 dinitroazetidine) from EpicentRx and Azelnidipine (CAS number 123524-52-7) or pharmaceutically acceptable salts thereof.

Various CD47 blockades that may be used are found in Table 1 of Zhang, et al., (2020), Frontiers in Immunology vol 11, article 18, and in Table 3 below.

TABLE 3

| Company | Approach | Agent/Program |
|---|---|---|
| Akesobio Australia | CD47 mAb | AK117 |
| Pty Ltd | | |
| Arch Oncology | CD47 mAb | AO-176 |
| (Tioma Therapeutics) | | |
| Elpiscience | CD47 | ES004 |
| Biopharma Inc. | | |
| EpicentRx | Small molecule | RRx-001 |
| | inhibitor of | (1-bromoacetyl-3,3 |
| | dinitroazetidine | dinitroazetidine) |
| | hypoxia sensor to | |
| | downregulate | |
| | CD47/SIRPα | |
| ImmuneOncia | CD47 mAb human | IMC-002 |
| Therapeutics | | |
| Innovent Biologics | CD47 mAb | IBI-188 (CD47 mAb) |
| | CD47/PD-L1 | IBI-322 (Bispecific) |
| | bispecific mAb | |
| OSE | SIRPα mAb | BI 765063 (OSE-172) |
| Zai Lab | CD47 mAb | ZL-1201 |
| Alx Oncology | High-affinity | ALX148 |
| | SIRPα-Fc | |
| Gilead/Forty Seven | CD47 mAb | Magrolimab |
| | SIRPα mAb | FSI-189 |
| I-Mab Biopharma | CD47 mAb | TJC4 |
| Jiangsu HengRui | CD47 mAb | SHR-1603 |
| Medicine Co., Ltd. | | |
| Surface Oncology | CD47 mAb human | SRF231 |
| Morphiex | CD47 targeting | MBT-001 |
| | phosphorodiamidate | |
| | morpholino | |
| | oligomers | |

Therapeutically effective doses of an anti-CD47 antibody or other protein CD47 inhibitor may be a dose that leads to sustained serum levels of the protein of about 40 μg/ml or more (e.g., about 50 ug/ml or more, about 60 ug/ml or more, about 75 ug/ml or more, about 100 ug/ml or more, about 125 ug/ml or more, or about 150 ug/ml or more). Therapeutically effective doses or administration of a CD47 blockade, such as an anti-CD47 antibody or SIRPα fusion protein or small molecule, include, for example, amounts of 0.05-10 mg/kg (agent weight/subject weight), such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits. Therapeutically effective doses of a small molecule CD47 blockade such as those disclosed herein also, for example, include 0.01 mg/kg to 1,000 mg/kg and any subrange or value of mg/kg therein such as 0.01 mg/kg to 500 mg/kg or 0.05 mg/kg to 500 mg/kg, or 0.5 mg/kg to 200 mg/kg, or 0.5 mg/kg to 150 mg/kg, or 1.0 mg/kg to 100 mg/kg, or 10 mg/kg to 50 mg/kg.

According to certain aspects, the anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example, as described in U.S. Pub. No. 20100239579).

Additional Agents

The methods of the present invention, which include administration of a radiotherapeutic and a CD47 blockade, may further include administration of one or more additional therapeutic agents. According to certain aspects, the additional agent(s) may be relevant for the disease or condition being treated. Such administration may be simultaneous, separate or sequential with the administration of the radiotherapeutic and CD47 blockade. For simultaneous administration, the agents may be administered as one composition, or as separate compositions, as appropriate.

Exemplary additional therapeutic agents include at least chemotherapeutic agents, anti-inflammatory agents, immunosuppressive agents, immunomodulatory agents, or a combination thereof. Moreover, the one or more further therapeutic agents may include an antimyeloma agent, such as dexamethasone, doxorubicin, bortezomib, lenalidomide, prednisone, carmustine, etoposide, cisplatin, vincristine, cyclophosphamide, and thalidomide.

According to certain aspects of the present invention, the methods may further include administration of a cytokine such as granulocyte colony-stimulating factor (GCSF) after administration of the radiotherapeutic and/or CD47 blockade. The GCSF may be administered, for example, 7, 8, 9, 10, or 11 days after administration of the radiolabeled CD33 targeting agent.

Exemplary chemotherapeutic agents include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, GCSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza®) and release of transcriptional repression (ATRA) therapies can also be combined with antibodies of the invention.

According to certain aspects, the chemotherapeutic agent may be selected from the group consisting of taxanes (e.g., paclitaxel (Taxol®), docetaxel (Taxotere®), modified paclitaxel (e.g., Abraxane and Opaxio®), doxorubicin, sunitinib (Sutent®), sorafenib (Nexavar®), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio)).

According to aspects of the presently disclosed invention, the chemotherapeutic agent is selected from 5-fluorouracil (5-FU), leucovorin, irinotecan, or oxaliplatin. According to certain aspects, the chemotherapeutic agent is 5-fluorouracil, leucovorin and irinotecan (FOLFIRI). According to other aspects, the chemotherapeutic agent is 5-fluorouracil, and oxaliplatin (FOLFOX).

According to aspects of the presently disclosed invention, the chemotherapeutic agent is selected from taxanes (e.g., docetaxel or paclitaxel) or a modified paclitaxel (e.g., Abraxane or Opaxio), doxorubicin), capecitabine and/or bevacizumab (Avastin®) for the treatment of breast cancer; therapies with carboplatin, oxaliplatin, cisplatin, paclitaxel, doxorubicin (or modified doxorubicin (Caelyx® or Doxil®)), or topotecan (Hycamtin®) for the treatment of ovarian cancer; therapies with a multi-kinase inhibitor, MKI, (Sutent, Nexavar, or AMG 706) and/or doxorubicin for the treatment of kidney cancer; therapies with oxaliplatin, cisplatin and/or radiation for the treatment of squamous cell carcinoma; and therapies with taxol and/or carboplatin for the treatment of lung cancer.

The therapeutic agents may be administered according to any standard dose regime known in the field. For example, therapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

Exemplary anti-inflammatory agents may be selected from a steroidal drug and a NSAID (nonsteroidal anti-inflammatory drug). Other anti-inflammatory agents may be selected from aspirin and other salicylates, Cox-2 inhibitors (such as rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies, anti-IL15 antibodies, anti-IL15R antibodies, anti-CD4 antibodies, anti-CD11a antibodies (e.g., efalizumab), anti-alpha4/beta-1 integrin (VLA4) antibodies (e.g natalizumab), CTLA4-1 g for the treatment of inflammatory diseases, prednisolone, prednisone, disease modifying antirheumatic drugs (DMARDs) such as methotrexate, hydroxychloroquine, sulfasalazine, pyrimidine synthesis inhibitors (such as leflunomide), IL-1 receptor blocking agents (such as anakinra), TNF-α blocking agents (such as etanercept, infliximab, and adalimumab) and similar agents.

Exemplary immunosuppressive and/or immunomodulatory agents include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents.

According to certain aspects of the presently disclosed invention, the additional therapeutic agents may include an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

According to certain aspects of the presently disclosed invention, the additional therapeutic agents may include allopurinol, administered at a dose of 300-600 mg/day orally starting on day 1 of the treatment period and continuing for at least 7 days after the CD33 targeting agent. Prophylactic antibiotics and antifungal therapies may be included for those patients who have an absolute neutrophil count of less than 500/ul. Analgesics and antihistamines may also be included prior at administration of the CD33 targeting agent by infusion to reduce infusion-related reactions.

The additional therapeutic agents may be administered according to any standard dose regime known in the field. For example, therapeutic agents may be administered at concentrations in the range of 1 to 500 mg/m², the amounts being calculated as a function of patient surface area (m²). For example, exemplary doses of paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

Without limitation, the following aspects of the invention are also provided by this disclosure:

Aspect 1: A therapeutic composition for the treatment of a cancer in a mammalian subject such as a human, the composition including: a radiotherapeutic agent, such as a radiolabeled cancer-targeting agent, such as a radiolabeled antigen targeting agent targeting a preselected cancer-associated antigen such as any of those disclosed herein, such as a radiolabeled antibody targeting a preselected cancer-associated antigen such as any of those disclosed herein; and a CD47 blockade.

Aspect 2: The composition according to aspect 1, wherein the radiotherapeutic agent includes a radiolabeled CD33, DR5, 5T4, HER2, HER3, or TROP2 targeting agent, such as a radiolabeled anti-CD33, anti-DR5, anti-5T4, anti-HER2, anti-HER3, or anti-TROP2 monoclonal antibody, or a radiolabeled antigen-binding fragment of any of the preceding monoclonal antibodies.

Aspect 3: The composition according to any preceding aspect, wherein the radiotherapeutic agent includes a radiolabel selected from ¹³¹I, ¹²⁵I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

Aspect 4: The composition according to any preceding aspect, wherein the radiotherapeutic includes a CD33 targeting agent selected from radiolabeled lintuzumab, gemtuzumab, vadastuximab, or a combination thereof, such as actinium-225 or lutetium-177 labeled lintuzumab, gemtuzumab, vadastuximab, or a combination thereof.

Aspect 5: The composition according to any preceding aspect, wherein the radiotherapeutic includes a radiolabeled DR5 targeting agent selected from radiolabeled mapatumumab, conatumumab, lexatumumab, tigatuzumab, drozitumab, LBY-135, or a combination thereof, such as any of the aforementioned targeting agents or any combination thereof radiolabeled with actinium-225 or lutetium-177.

Aspect 6: The composition according to any preceding aspect, wherein the radiotherapeutic includes a radiolabeled 5T4 targeting agent selected from radiolabeled MED10641, ALG.APV-527, Tb535, H6-DM5, ZV0508, or a combination thereof, such as any of the aforementioned targeting agents or any combination thereof radiolabeled with actinium-225 or lutetium-177.

Aspect 7: The composition according to any preceding aspect, wherein the radiotherapeutic includes a radiolabeled HER3 targeting agent selected from radiolabeled patritumab, seribantumab, lumretuzumab, elgemtumab, AV-203, GSK2849330, or a combination thereof, such as any of the aforementioned targeting agents or any combination thereof radiolabeled with actinium-225 or lutetium-177.

Aspect 8: The composition according to any preceding aspect, wherein the effective amount of the actinium-225 labeled radiotherapeutic includes a radiation dose of 0.1 to 10 μCi/kg body weight of the subject and a protein dose of less than 10 mg/kg body weight of the subject.

Aspect 9: The composition according to any preceding aspect, wherein the effective amount of the actinium-225 labeled radiotherapeutic includes a radiation dose of 0.1 to 2 μCi/kg body weight of the subject and a protein dose of less than 5 mg/kg body weight of the subject.

Aspect 10: The composition according to any preceding aspect, wherein the CD47 blocking agent includes a monoclonal antibody that prevents CD47 binding to SIRPα.

Aspect 11: The composition according to any preceding aspect, wherein the CD47 blocking agent includes magrolimab, lemzoparlimab, AO-176, TTI-621, TTI-622, ALX148, RRx-001, Azelnidipine, any CD47 blockade disclosed herein, or any combination thereof.

Aspect 12: The composition according to any preceding aspect, wherein the effective amount of the CD47 blocking agent is 0.05 to 5 mg/kg (agent weight/body weight).

Aspect 13: The composition according to any preceding aspect, wherein the radiotherapeutic includes m¹⁵Ac-lintuzumab having a radiation dose of 0.1 to 2 μCi/kg body weight of the subject and a protein dose of less than 5 mg/kg body weight of the subject.

Aspect 14: The composition according to any preceding aspect, wherein the cancer is a solid tumor cancer.

Aspect 15: The composition according to any one of aspects 1-14, wherein the cancer is a hematological cancer.

Aspect 16: The composition according to aspect 15, wherein the hematological cancer is a myeloid malignancy.

Aspect 17: The composition according to aspect 15, wherein the hematological cancer includes multiple myeloma, acute myelogenous leukemia, chronic myelogenous leukemia, myelodysplastic syndrome, or myeloproliferative neoplasm.

Aspect 18: The composition according to any one of aspects 14-17, wherein the hematological cancer is relapsed/refractory acute myeloid leukemia.

Aspect 19: The composition according to any preceding aspect, wherein the cancer is a CD33 positive, DR5 positive, 5T4 positive, HER2, HER3, and/or TROP2 positive cancer.

Aspect 20: The composition according to aspect 19, wherein the CD33 positive cancer includes cells expressing CD33, wherein the CD33 expressing cells include myeloblast cells or malignant plasmacytes.

Aspect 21. The composition according to any preceding aspect, further including at least one pharmaceutically acceptable excipient.

Aspect 22: A method for treating a cancer in a mammalian subject, such as a human, the method including administering a composition according to any one of aspects 1 to 21.

Aspect 23: A method for treating a cancer in a mammalian subject, such as a human, the method including administering (i) a radiolabeled cancer-targeting agent, such as any of those disclosed herein, such as a radiolabeled antigen-targeting agent targeting a preselected cancer-associated antigen such as any of those disclosed herein, such as a radiolabeled antibody targeting a preselected cancer-associated antigen such as any of those disclosed herein; and (ii) a CD47 blockade.

Aspect 24: The method according to aspect 22 or 23, wherein the radiotherapeutic agent includes

an anti-CD33 monoclonal antibody or a CD33-binding fragment thereof, and the cancer is a hematological disease or disorder selected from one or more of multiple myeloma, acute myelogenous leukemia, chronic myelogenous leukemia, myelodysplastic syndrome, and myeloproliferative neoplasm, or a solid tumor cancer such as any of those disclosed herein; or

an anti-5T4 monoclonal antibody or a 5T4-binding fragment thereof, and the cancer is colorectal cancer, gastric cancer, ovarian cancer, non-small cell lung carcinoma, head and neck squamous cell cancer, pancreatic cancer, renal cancer, or any combination thereof; or

an anti-DR5 monoclonal antibody or a DR5-binding fragment thereof, and the cancer is breast cancer, triple negative breast cancer, ovarian cancer, or prostate cancer;

an anti-HER2 monoclonal antibody or a HER2-binding fragment thereof, and the cancer is pancreatic cancer, lung cancer, head and neck cancer, breast cancer, gastric cancer, colorectal cancer, esophageal cancer, or ovarian cancer;

an anti-HER3 monoclonal antibody or a HER3-binding fragment thereof, and the cancer is pancreatic cancer, lung cancer, head and neck cancer, breast cancer, gastric cancer, colorectal cancer, esophageal cancer, or ovarian cancer; or

an anti-TROP2 monoclonal antibody or a TROP2-binding fragment thereof, and the cancer is pancreatic cancer, lung cancer, head and neck cancer, breast cancer, gastric cancer, colorectal cancer, esophageal cancer, or ovarian cancer.

Aspect 25: The method according to aspect 22 or 23, wherein the radiotherapeutic agent includes a radiolabeled CD33, DR5, 5T4, HER2, HER3, or TROP2 targeting agent, such as a radiolabeled anti-CD33, anti-DR5, anti-5T4, anti-HER2, anti-HER3, or anti-TROP2 monoclonal antibody.

Aspect 26: The method according to any one of aspects 22-25, wherein the radiotherapeutic agent includes a radiolabel selected from ¹³¹I, ¹²⁵I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

Aspect 27: The method according to any one of aspects 22-26, wherein the radiotherapeutic includes a CD33 targeting agent selected from radiolabeled lintuzumab, gemtuzumab, vadastuximab, or a combination thereof, such as actinium-225 or lutetium-177 labeled lintuzumab, gemtuzumab, vadastuximab, or a combination thereof.

Aspect 28: The method according to any one of aspects 22-27, wherein the radiotherapeutic includes a radiolabeled DR5 targeting agent selected from radiolabeled mapatumumab, conatumumab, lexatumumab, tigatuzumab, drozitumab, LBY-135, or a combination thereof, such as any of the aforementioned targeting agents or any combination thereof radiolabeled with actinium-225 or lutetium-177.

Aspect 29: The method according to any one of aspects 22-28, wherein the radiotherapeutic includes a radiolabeled 5T4 targeting agent selected from radiolabeled MED10641, ALG.APV-527, Tb535, H6-DM5, ZV0508, or a combination thereof, such as any of the aforementioned targeting agents or any combination thereof radiolabeled with actinium-225 or lutetium-177.

Aspect 30: The method according to any one of aspects 22-29, wherein the radiotherapeutic includes a radiolabeled HER3 targeting agent selected from radiolabeled patritumab, seribantumab, lumretuzumab, elgemtumab, AV-203, GSK2849330, or a combination thereof, such as any of the aforementioned targeting agents or any combination thereof radiolabeled with actinium-225 or lutetium-177.

Aspect 31: The method according to any one of aspects 22-29, wherein the effective amount of the actinium-225 labeled radiotherapeutic includes a radiation dose of 0.1 to 10 μCi/kg body weight of the subject and a protein dose of less than 10 mg/kg body weight of the subject.

Aspect 32: The method according to aspect 31, wherein the effective amount of the actinium-225 labeled radiotherapeutic includes a radiation dose of 0.1 to 2 μCi/kg body weight of the subject and a protein dose of less than 5 mg/kg body weight of the subject.

Aspect 33: The method according to any one of aspects 22-30, wherein the CD47 blocking agent includes a monoclonal antibody that prevents CD47 binding to SIRPα.

Aspect 34: The method according to any one of aspects 22-30, wherein the CD47 blocking agent includes magrolimab, lemzoparlimab, AO-176, TTI-621, TTI-622, ALX-148, RRx-001, Azelnidipine, any CD47 blockade disclosed herein, or any combination thereof.

Aspect 35: The method according to any one of aspects 22-34, wherein the effective amount of the CD47 blocking agent is 0.05 to 5 mg/kg (agent weight/body weight).

Aspect 36: The method according to any one of aspects 22-35, wherein the radiotherapeutic includes m¹⁵Ac-lintuzumab having a radiation dose of 0.1 to 2 μCi/kg body weight of the subject and a protein dose of less than 5 mg/kg body weight of the subject.

Aspect 37: The method according to any one of aspects 22-36, wherein the cancer is a solid tumor cancer.

Aspect 38: The method according to any one of aspects 22-37, wherein the cancer is a hematological cancer.

Aspect 39: The method according to aspect 38, wherein the hematological cancer is a myeloid malignancy.

Aspect 40: The method according to aspect 38, wherein the hematological cancer includes multiple myeloma, acute myelogenous leukemia, chronic myelogenous leukemia, myelodysplastic syndrome, or myeloproliferative neoplasm.

Aspect 41: The composition according to any one of aspects 38-40, wherein the hematological cancer is relapsed/refractory acute myeloid leukemia.

Aspect 42: The composition according to any one of aspects 22-41, wherein the cancer is a CD33 positive, DR5 positive, 5T4 positive, HER2, HER3, and/or TROP2 positive cancer.

Aspect 43: The composition according to aspect 42, wherein cancer includes CD33 positive cancers including one or both of CD33 positive myeloblast cells and CD33 positive malignant plasmacytes.

Aspect 44. Use of a therapeutically active radiolabeled cancer-targeting targeting agent such as a radiolabeled agent targeting a cancer-associated antigen or otherwise targeting cancer cells, such as any of those disclosed herein, in the preparation of a medicament for the treatment of a cancer or a precancerous proliferative disorder, such as a hematological malignancy or a solid cancer, such as any of those disclosed herein, in a mammalian subject such as a human patient, in combination with a CD47 blockade, such as any of those disclosed herein.

Aspect 45. Use of a CD47 blockade/blocking agent, such as any of those disclosed herein, in the preparation of a medicament for the treatment of a cancer or a precancerous proliferative disorder, such as a hematological malignancy or a solid cancer, such as any of those disclosed herein, in a mammalian subject such as a human patient, in combination with a therapeutically active radiolabeled cancer targeting agent targeting a cancer-associated antigen or otherwise targeting cancer cells, such as any of those disclosed herein.

Aspect 46. Use of a therapeutically active radiolabeled cancer targeting agent targeting a cancer-associated antigen or otherwise targeting cancer cells, such as any of those disclosed herein, in combination with a CD47 blockade, such as any of those disclosed herein, for the treatment of a cancer or a precancerous proliferative disorder, such as hematological malignancy or a solid cancer, such as any of those disclosed herein, in a mammalian subject such as a human patient.

Aspect 47. Any of the preceding aspects, wherein the radiolabeled targeting agent includes a targeting agent chemically conjugated to a chelator, wherein the chelator chelates a radionuclide such as any of those disclosed herein.

Aspect 48. Preceding aspect 47, wherein the chelator includes DOTA or a DOTA derivative.

Aspect 49. Any of the preceding aspects, wherein the radionuclide is ¹⁷⁷Lu, ²²⁵Ac, ¹³¹I, or ⁹⁰Y.

In one variation, the various aspects and embodiments of the invention are not part of a cellular therapy, such as an engineered cell therapy, such as CAR-T therapy, and are not used in combination or conjunction with a cell therapy, such as a genetically engineered cell therapy, such as CAR-T therapy. Thus, in one variation, the methods of treatment of the invention do not include a cell therapy, such as a genetically engineered cell therapy, such as CAR-T therapy.

In one variation of the various aspects and embodiment of the invention, the CD47 blockade agent, such as anti-CD47 mAb or anti-SIRPα mAb or SIRPα-Fc fusion protein, is not radiolabeled. In another variation of the various aspects and embodiments of the invention, the CD47 blockade agent and the radiolabeled targeting agent or radiotherapeutic are separate and discrete molecules, i.e., not parts of the same molecule.

Advantageously, CD47 blockade increases the overall tolerability and survivability of a mammalian subject to the radiation dose(s) delivered by the radiolabeled agent (and any external radiation and/or brachytherapy) without substantially reducing lethality of the combined treatment toward the target cancer cells (or target precancerous disorder cells), thereby permitting higher, more effective radiation doses to be employed, and/or more frequent dosing, and/or longer courses of treatment than could be employed without the CD47 blockade.

### EXAMPLES

### Example 1: Production of Radiolabeled Targeting Agent

A targeting agent such as an antibody or other protein or peptide may, for example, be labeled with a radionuclide, such as ¹³¹I or ²²⁵Ac, according to the procedures described in any of U.S. Pat. Nos. 10,420,851, 9,603,954, International Pub. No. WO 2017/155937 and U.S. Provisional Patent Application No. 63/042,651 filed Dec. 9, 2019 and titled “Compositions and methods for preparation of site-specific radioconjugates.”

Radiolabeling: The antibody may be conjugated to a linker, such as any of the linkers described in the above indicated patent applications. An exemplary linker includes at least dodecane tetraacetic acid (DOTA), wherein a goal of the conjugation reaction is to achieve a DOTA-antibody ratio of 3:1 to 5:1. Chelation with the radionuclide, such as ¹⁷⁷Lu, ⁹⁰Y, or ²²⁵Ac may then be performed and efficiency and purity of the resulting radiolabeled antibody, such as an anti-CD33 antibody, may be determined by HPLC and iTLC.

An exemplary labeling reaction for ²²⁵Ac is as follows: A reaction including 15 μl 0.15M NH₄OAc buffer, pH=6.5 and 2 μL (10 μg) DOTA-anti-CD33 (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 min with shaking at 100 rpm. At the end of the incubation period, 3 μL of a 1 mM DTPA solution may be added to the reaction mixture and incubated at room temperature for 20 min to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-CD33 through separating 225Ac-labeled anti-CD33 (²²⁵Ac-DOTA-anti-CD33) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: An exemplary radiolabeled targeting agent, such as ²²⁵Ac-DOTA-antibody, may be purified either on PD10 columns pre-blocked with 1% HSA or on Vivaspin centrifugal concentrators with a 50 kDa MW cut-off with 2×1.5 mL washes, 3 min per spin. HPLC analyses of the ²²⁵Ac-DOTA-antibody after purification may be conducted using a Waters HPLC system equipped with flow-through Waters UV and Bioscan Radiation detectors, using a TSK3000SW XL column eluted with PBS at pH=7.4 and a flow rate of 1 ml/min.

### Example 2: Specificity and Stability of CD33 ARC

Lintuzumab conjugated with Actinium-225 (²²⁵Ac) was tested for cytotoxicity against specific cell types which express CD33. For example, suspensions of HL60 (leukemia cells) were incubated with various doses of radiolabeled lintuzumab (lintuzumab-Ac²²⁵), and the dose at which 50% of the cells were killed (LD₅₀) was found to be 8 pCi per mL of cell suspension.

In studies to access the reactivity of the radiolabeled lintuzumab with peripheral blood and bone marrow cells from nonhuman primate and human frozen tissues, the radiolabeled lintuzumab showed reactivity with mononuclear cells only, demonstrating specificity. Moreover, in studies to determine the stability of the radiolabel on the antibody, 10 normal mice (8-week old Balb/c female mice from Taconic, Germantown, N.Y.) were injected in the tail with 300 nCi radiolabeled lintuzumab (in 0.12 ml). Serum samples taken over a 5-day period showed that the Actinium-225 remained bound to the lintuzumab, demonstrating the stability of the radiolabel on the antibody in vivo.

A maximum tolerated dose (MTD) of a single injection of the radiolabeled lintuzumab was determined to be 3 μCi/kg patient weight. As a split dose (e.g., 2 equal doses administered 4-7 days apart), the MTD was determined to be 2 μCi/kg per dose, or 4 μCi/kg total. This data was determined by injections into patients with relapsed/refractory AML: 21 patients were injected with increasing doses of the radiolabeled lintuzumab—0.5 μCi/kg to 4 μCi/kg. Determination of MTD was based on the severity of the adverse effects observed at each dose level. Anti-leukemic effects included elimination of peripheral blood blasts in 13 of 19 evaluable patients. Twelve of 18 patients who were evaluable at 4 weeks following treatment had reductions in bone marrow blasts, including nine with reductions ≥50%. Three patients treated with 1 μCi/kg, 3 μCi/kg and 4 μCi/kg respectively had ≤5% blasts after therapy.

### Example 3: Human Maximal Tolerated Dose and Efficacy of CD33 ARC

A maximum tolerated dose (MTD) of fractionated doses of lintuzumab-Ac²²⁵ followed by Granulocyte Colony Stimulating factor (GCSF) support in each cycle may be determined using a dosing cycle of approximately 42 days. A cycle starts with administration of a fractionated dose of Lintuzumab-Ac²²⁵ on Day 1 followed by the administration of GCSF on Day 9 and continuing GCSF per appropriate dosing instructions until absolute neutrophil count (ANC) is greater than 1,000, which is expected to occur within 5-10 days. On Days 14, 21, 28, 35 and 42 peripheral blood may be assessed for paraprotein burden. A bone marrow aspirate will be performed to assess plasmocyte infiltration on Day 42. If a response is a partial response or better but less than a complete response on Day 42, and the patient remains otherwise eligible, the patient will be re-dosed in a new cycle at the same dose level no sooner than 60 days after Day 1 of the first cycle. In absence of dose limiting toxicities, cycles will continue using the above-described algorithm until the patient has received a cumulative dose of 4 μCi/kg of lintuzumab-Ac²²⁵.

### Example 4: Syngeneic Mouse Model for 5T4 Targeting Agents

A syngeneic mouse model may be used to explore targeting 5T4 in a model where the antibody can also react with 5T4 expressed on normal tissues. Such a model provides the opportunity to observe any toxicities that may arise through targeting this protein with a radioisotope warhead.

Woods (Woods, A. M. et al. (2002) Biochem. J. 366, 353-365) reported discovery of an antibody (9A7) that is reactive to mouse 5T4 and was used to screen mouse tumor lines for 5T4 expression (see Table 4; taken from Woods, 2002). Of the cell lines reported to be positive for 5T4 expression, the EMT6 mammary adenocarcinoma cell line has high levels of 5T4 expression, is readily available for purchase from commercial sources to perform experiments. Moreover, this cell line has been reported to be sensitive to radiation. Certain mouse 5T4-reactive antibodies are available, including B3F1 (Southgate, T. D. et al. (2010) PLoS One 5, e9982). This antibody exhibits strong binding to 5T4 in ELISA, FACS, and Western blot assays and is suitable as a targeting agent in preclinical proof of concept studies. Therefore, the B3F1 anti-mouse 5T4 antibody will be utilized for radiolabeling to target the 5T4-expressing tumor cell line EMT6.

Experimental plan: A exemplary experimental plan includes conjugation of the 5T4 antibody B3F1 with the chelator DOTA, following by radiolabeling with ¹¹¹In or ²²⁵Ac. Specific activity, efficiency of labeling, and stability of the radiolabeled antibody can be determined as set forth in Examples 1 and 2.

TABLE 4

| Cell Line | Origin | Flow cytometry |
|---|---|---|
| A9 neo | Lung fibroblast L cells | − |
| A9-m5T4 | Lung fibroblast L cells | + + + + |
| B16 F10 Neo | Melanoma | − |
| B16 F10-m5T4 | Melanoma | + + |
| EMT6 | Mammary adrenocarcinoma | + + + |
| C1271 | Mammary carcinoma | + + + |
| Clone M3 | Melanoma | − |
| EL4 | Lymphoma | − |
| KLN-205 | Squamous cell lung carcinoma | +/− |
| JC | Breast adenocarcinoma | − |
| LL/2 | C57BL Lewis lung carcinoma | − |
| Mosec | Ovarian carcinoma | − |
| Nulli 2A | Embryonic carcinoma | + |
| 129 ES | Embryonic stem cell | − |
| CL-S1 | BALB/c mammary pre-neoplastic | +/− |
| | alveolar nodules | |
| CMT-93 | Rectal carcinoma | − |

An in vitro cell killing assay may be performed with the ²²⁵Ac radiolabeled B3F1 antibody. EMT6 cells may be used as a positive control for cells that express 5T4 and will be exposed to a dilution series of 225Ac-labeled DOTA-B3F1 and unlabeled DOTA-B3F1 for 1 hour. Cell viability can be measured using an XTT assay as described hereinabove. If desired, a cell line that does not express 5T4 such as LL/2 cells (see Table 4, Source—Woods, 2002) can be used as a negative control.

Table 4 shows a Fluorescent Activated Cell Sorting (FACS) analysis of the 9A7 antibody against a panel of murine cell lines, wherein 105 cells of each line were stained with 9A7. The last column indicates the relative reactivity of 9A7 against the panel of cell lines, wherein the mammary cell line EMT6 is highly reactive and the lung carcinoma cell line LL/2 is non-reactive.

Biodistribution experiments: An ¹¹¹In labeled B3F1 antibody can be used in a first round of biodistribution experiments performed with tumor-free BALB/c mice to evaluate any binding of the antibody to normal tissues and to calculate absorbed dose of radiation to organs. A second round of biodistribution experiments can be performed using BALB/c mice bearing EMT6 tumors to evaluate specific targeting of antibody to the 5T4-expressing tumor and to calculate absorbed dose of radiation to the tumor and to other organs.

Following biodistribution experiments, tumor-bearing mice can be treated with escalating single doses of ²²⁵Ac-DOTA-B3F1 to establish the maximum tolerated dose (MTD) of the antibody. The range of doses may be from 50 nCi to 400 nCi. Tolerability of the antibody can be determined through measurements of body weight, behavior, and blood chemistry/counts.

### Example 5: Xenograft Mouse Model for 5T4 Targeting Agent

Xenograft mouse models may be utilized to determine if a therapeutic targeting agent has an effect on human derived cancerous cells. However, unless the targeting agent cross-reacts with the mouse target, it primarily only provides information about the cell-killing ability of the agent on the xenograft cells and may not provide information regarding on-target but off-tumor effects.

Numerous anti-human 5T4 therapies have been developed for 5T4-expressing cancers, some of which are summarized in Table 1. The original description of an anti-5T4 antibody sequence was provided by Hole & Stem (Hole, 1988). An antibody for use as an 5T4 targeting agent according to the presently disclosed invention, such as in preclinical studies, may be produced using the sequence provided by Hole & Stem. Alternatively, other 5T4 antibodies that may be used include the following antibodies or the antibody portions of the following: Medimmune/AstraZeneca (MED10641), Aptevo Therapeutics/Alligator Bioscience (ALG.APV-527), Biotecnol/Chiome Bioscience (Tb535), Guangdong Zhongsheng Pharmaceuticals (H₆-DM5), and Zova Biotherapeutics (ZV0508). Additional antibodies that are bispecific or are available as antibody drug conjugates are listed in Table 1 and provide additional 5T4 targeting agents, i.e., the 5T4 specific binding portions.

The Medimmune/Astrazeneca antibody includes an engineered cysteine, which can be used for site-specific conjugation of DOTA and subsequent chelation with a radioisotope, such as described in U.S. Provisional Patent Application Nos. 62/945,383 filed Dec. 9, 2019 and 63/119,093 filed Nov. 30, 2020 each titled “Compositions and methods for preparation of site-specific radioconjugates,” incorporated by reference herein.

Tumor Dose

Biodistribution studies may be performed in mice with 4T1 tumors to establish the normal tissue distribution and dosimetry profile of the DR5-targeting ARC and to confirm the selective uptake of the radiolabeled MD5-1 antibody to the tumor. ¹¹¹In will again be used as a surrogate for ²²⁵Ac due to the similar radiochemical properties of the two isotopes, and the increased sensitivity and reliability of detection of ¹¹¹In-radiolabeled agents in vivo due to the gamma-emission from this isotope that does not occur with ²²⁵Ac. Five groups of 4 female mice (ages 6-8 weeks) each will be injected with ¹¹¹In-labeled MD5-1 and one group of mice will be euthanized at each of the following time points: 4, 24, 48, 96, and 168 hours. Organs (liver, lung, kidney, spleen, brain, stomach, muscle, and tumor) may then be harvested and gamma counts measured. These measurements will be used for dosimetry calculations in which the absorbed dose of radiation to each organ is determined, including the dose delivered to the tumor.

### Example 7: Determine MTD and Single Agent Activity of 225Ac-MD5-1

Six (6) groups with 6 mice per group, with established subcutaneous 4T1 tumors (˜150-200 mm3) will be injected with unlabeled MD5-1 (500 ng) or a dose escalation of ²²⁵Ac-MD5-1 (50, 100, 200, 400, 500 nanoCurie (nCi), 500 ng total antibody) to identify the maximum tolerated dose (MTD), which is defined as the highest administered activity that allows survival of all treated mice without resulting in >20% weight loss. Bodyweights and tumor measurements may be taken twice weekly for the 6-week duration of the study, beginning at animal arrival to the Invicro facility. Serum chemistry (Alanine Aminotransferase, Alkaline Phosphatase, Total bilirubin, Blood Urea Nitrogen, Calcium, Phosphorus, Total Protein, Albumin, Globulin, Albumin/Globulin Ratio, Amylase, Glucose, Total Cholesterol, Lipase) and complete blood counts (CBC) will be evaluated in animals on-study at baseline, week 3, and at the terminal time point. Humane euthanasia criteria include a decrease in body weight of >20%, or an increase in body weight due to ascites of >10%. Any signs of pain or distress may also be considered.

Animal health measurements and observations can be used to determine the MTD. Observations on tumor volume and survival may be recorded and considered with the toxicity/tolerability profile, to determine the MTD of ²²⁵Ac-MD5-1. During this experimental stage, preliminary anti-tumor efficacy can be determined by tumor measurements, and survival used as a proxy for lung metastases, as has been previously reported (Demaria, S. et al. (2005) Clin. Cancer Res. 11, 728-34).

### Example 8 Combination of HER2 Targeting ARC and CD47 Blocking Antibody in Human Solid Tumor Cancer Model

These studies examined the effects of combining a HER2 specific targeting ARC and a CD47 blocking antibody on human HER2-expressing ovarian cancer cell line SK-OV3.

The anti-HER2 antibody Trastuzumab was conjugated with p-SCN-DOTA and radiolabeled with 225Ac or 177Lu. The biological activity of both radioconjugates was evaluated using human recombinant HER2 and receptor positive tumor cell lines. The cytotoxic effect of radioconjugates and the ability to upregulate calreticulin (CRT) was evaluated using XTT assay and flow cytometry, respectively, on the SK-OV3 cells. To evaluate the effect of anti-HER2 ARC and CD47 antibody combination in vitro, a flow cytometry macrophage phagocytosis assay was developed.

Results. The Trastuzumab ARCs have similar binding properties to native antibody and demonstrated specific cytotoxicity. Importantly, ARC-mediated CRT upregulation in HER2 expressing cells was demonstrated. Further, the combination of HER2 targeting ARC and CD47 blocking antibody enhanced in vitro macrophage-mediated tumor cell phagocytosis at a radiation dose below the maximum tested compared to the effect of each agent alone on phagocytosis.

These findings suggest that ARC mediated upregulation of CRT potentiates the pro-phagocytic signal and anti-CD47 mode of action, thereby enhancing antitumor activity.

Tumor xenograft studies examining the effect of ARC treatment in combination with CD47 blockade on tumor growth were also performed.

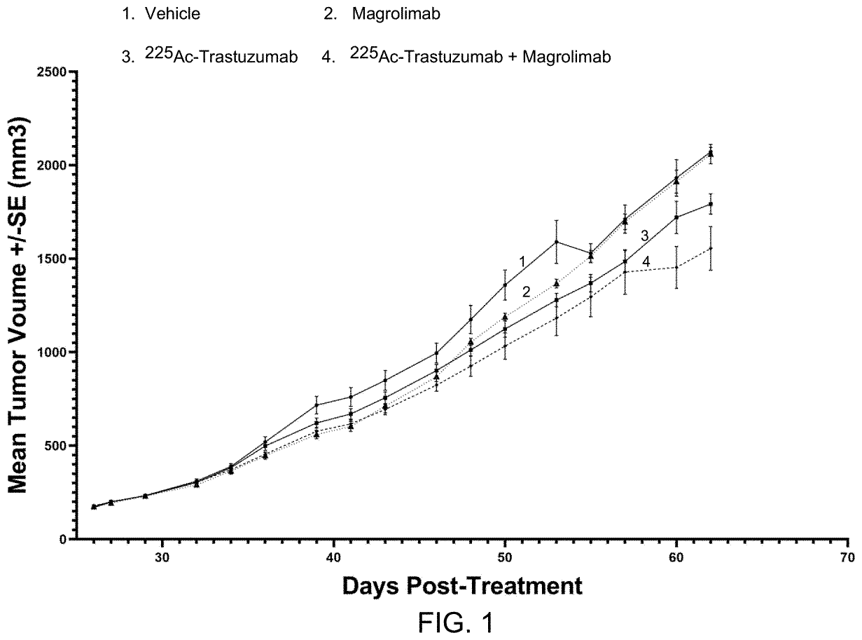

FIG. 1 is a graph showing the comparative effects on tumor growth of vehicle only (control), magrolimab alone (10 mg/kg), ²²⁵Ac-trastuzumab alone (0.025 μCi/animal), and the combination of magrolimab (10 mg/kg) and ²²⁵Ac-trastuzumab (0.025 μCi/animal), in an NGS mouse xenograft model using the SK-OV3 human ovarian cancer cell line. Each cohort consisted of eight animals.

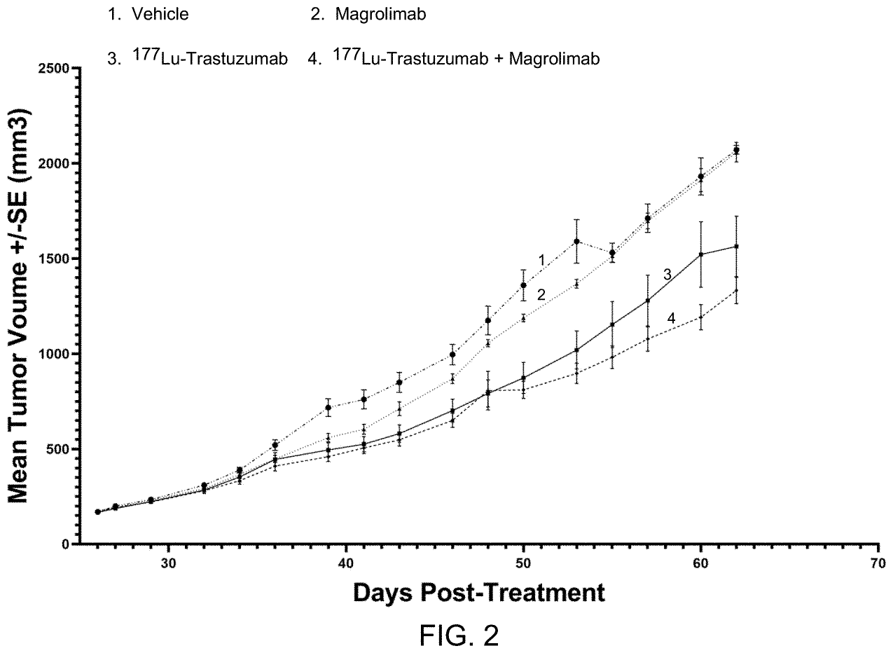

FIG. 2 is a graph showing the comparative effects on tumor growth of vehicle only (control), magrolimab alone (10 mg/kg), 177Lu-trastuzumab alone (25 μCi/animal), and the combination of magrolimab (10 mg/kg) and 177Lu-trastuzumab (25 μCi/animal), in an NGS mouse xenograft model using the SK-OV3 human ovarian cancer cell line. Each cohort consisted of eight animals.

### Example 9 Combination of CD33 Targeting ARC and CD47 Blocking Antibody in AML Models

These studies examined the effects of combining the anti-CD33 ARC armed with 225Ac or Lutetium-177 (177Lu) and a CD47 blocking antibody, using in vitro human AML model cell lines U937 and HL-60.

The anti-CD33 antibody Lintuzumab was conjugated with p-SCN-DOTA and radiolabeled with 225Ac or 177Lu. The biological activity of both radioconjugates was examined using human recombinant CD33 and receptor positive cell lines U937 and HL-60. The cytotoxic effect of the radioconjugates and the ability to upregulate calreticulin (CRT) were evaluated using XTT assay and flow cytometry, respectively, in the CD33 expressing cell lines. To assess the therapeutic combination of anti-CD33 ARC and CD47 antibody in vitro, a flow cytometry macrophage phagocytosis assay was used.

Results. The anti-CD33 ARCs have similar binding properties to native antibody and demonstrate specific cell cytotoxicity. ARC-mediated upregulation of cell surface CRT in both of the CD33 expressing AML cells was demonstrated. Further, the in vitro combination of CD33 targeting ARC and CD47 blocking antibody enhanced macrophage-mediated phagocytosis for both of the AML cell lines at a radiation dose less than the maximum tested, compared to the effect of each agent alone on phagocytosis.

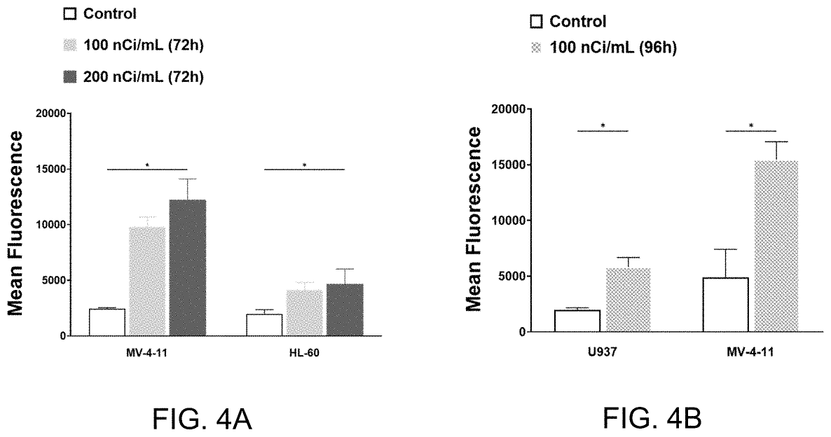

FIGS. 4A and 4B show that ²²⁵Ac-labeled lintuzumab induces an increase in cell surface calreticulin in human leukemia cell lines at different time points versus control untreated cells. Cell surface calreticulin (CRT) levels of AML cells (MV-4-11 and HL-60 in FIG. 4A and MV-4-11 and U937 in FIG. 4B) treated with 100 nCi/mL or 200 nCi/mL of ²²⁵Ac-labeled lintuzumab and of untreated control cells were detected by flow cytometry at 72 hours (FIG. 4A) or at 96 hours (FIG. 4B). Statistical analysis was performed using Two-Way ANOVA (*p<0.05).

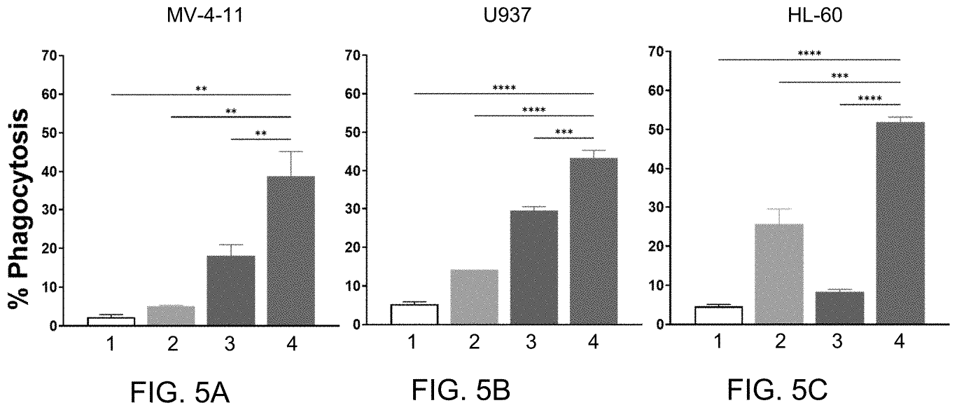

FIGS. 5A, 5B, and 5C show that combination treatment with ²²⁵Ac-labeled lintuzumab and an anti-CD47 antibody, B6.H12 (BioXCell, Lebanon, N.H., USA) enhances phagocytosis of three human leukemia cell lines. Target cells (MV-4-11 in FIG. 5A, U937 in FIG. 5B, and HL-60 in FIG. 5C) were treated with ²²⁵Ac-labeled lintuzumab for 96 hours. The cells were labeled with DiD and cocultured for 2 hours in the presence of the anti-CD47 mAb (1 pg/ml) with human macrophages labeled with DiO. The percentage of phagocytosis was measured by flow cytometry (macrophages DiO+/DiD+). Statistical analysis was performed using One-Way ANOVA (*p<0.05, **p<0.01, ***p<0.001 and ****p<0.0001). In each of FIGS. 5A-5C, bar 1 shows the results for non-specific IgG control, bar 2 show the results for the anti-CD47 mAb only, bar 3 shows the results for 100 nCi ²²⁵Ac-labeled lintuzumab only, and bar 4 shows the results for the combination of the anti-CD47 mAb and 100 nCi ²²⁵Ac-labeled lintuzumab.

These findings support a novel synergistic mechanism in which the CD33 ARC targeted radiation induces upregulation of CRT, thereby potentiating a pro-phagocytic innate immune response in combination with anti-CD47 blocking antibody.

### Example 10 Combination Effect of HER3 Targeting ARC and CD47 Blocking Antibody on Phagocytosis of Human BxPC3 Pancreatic Cells

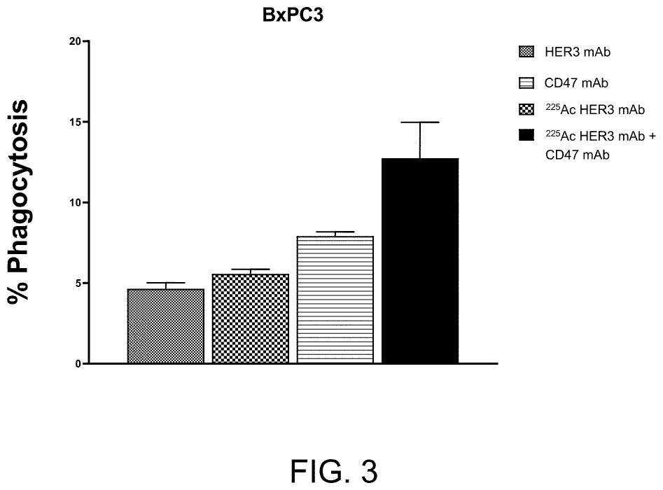

FIG. 3 is a graph showing the comparative effects on phagocytosis by human macrophages of BxPC3 human pancreatic cancer cell line (adenocarcinoma) cells of: a non-radiolabeled anti-human HER3 IgG monoclonal antibody AT-02 alone (“HER3 mAb”), an anti-human CD47 antibody alone (10 pg/mL; Clone B6.H12; BioXcell catalog no. BE0019-1; “CD47 mAb”), ²²⁵Ac-labeled AT-02 anti-HER3 mAb alone (100 nCi/mL; “²²⁵Ac-HER3 mAb”), and the combination of the anti-CD47 mAb (10 pg/mL) and the ²²⁵Ac-labeled AT-02 anti-HER3 mAb (100 nCi/mL). As shown in the figure, the combination prominently enhanced phagocytosis of BxPC3 cells versus any of the individual agents.

While various specific aspects and embodiments have been illustrated and described herein, it will be appreciated that various changes can be made without departing from the spirit and scope of the invention(s). Moreover, features described in connection with one aspect or embodiment of the invention may be used in conjunction with other aspects and embodiments of the invention, even if not explicitly exemplified in combination within.

What is claimed is:

1. A method for treating a cancer or precancerous proliferative disorder in a mammalian subject, comprising:
administering to a mammalian subject having the cancer or precancerous proliferative disorder an effective amount of one or more therapeutically radiolabeled cancer targeting agents; and
administering to the mammalian subject an effective amount of one or more CD47 blockades,
wherein the one or more CD47 blockades comprise one or more of an anti-CD47 antibody, an anti-SIRPα antibody, a SIRPα Fc fusion protein, a CD47 antisense phosphorodiamidate morpholino oligomer (PMO), and 1-bromoacetyl-3,3 dinitroazetidine or a pharmaceutically acceptable salt thereof.

2. The method of claim 1, wherein at least one of the radiolabeled cancer targeting agents is labeled with an alpha particle emitting radionuclide.

3. The method of claim 1, wherein at least one of the radiolabeled cancer targeting agents is labeled with a beta particle emitting radionuclide.

4. The method of claim 1, wherein the one or more radiolabeled cancer targeting agents comprises one or more of a monoclonal antibody against CD33 or an antigen-binding fragment thereof, a monoclonal antibody against DR5 or an antigen-binding fragment thereof, a monoclonal antibody against 5T4 or an antigen-binding fragment thereof, a monoclonal antibody against HER2 or an antigen-binding fragment thereof, or a monoclonal antibody against HER3 or an antigen-binding fragment thereof, a monoclonal antibody against TROP2 or an antigen-binding fragment thereof, and a monoclonal antibody against MUC1 or an antigen-binding fragment thereof.

5. The method of claim 4, wherein
the one or more radiolabeled cancer targeting agents comprise a composition of ²²⁵Ac-labeled antibody and non-radiolabeled antibody, the composition comprising a radiation dose of 0.1-2.0 μCi/kg body weight of the subject and a protein dose of 0.1-5.0 mg/kg body weight of the subject, and/or the CD47 blockade is administered at a total dose of 0.05-5.0 mg/kg body weight of the subject.

6. The method of claim 4, wherein the monoclonal antibody is an anti-CD33 antibody and the cancer or precancerous proliferative disorder is a solid tumor, osteosarcoma, multiple myeloma, acute myelogenous leukemia, chronic myelogenous leukemia, myelodysplastic syndrome, or myeloproliferative neoplasm.

7. The method of claim 4, wherein monoclonal antibody is an anti-5T4 antibody and the cancer or precancerous proliferative disorder is colorectal cancer, gastric cancer, ovarian cancer, non-small cell lung carcinoma, head and neck squamous cell cancer, pancreatic cancer, renal cancer, or any combination thereof.

8. The method of claim 4, wherein the monoclonal antibody is an anti-DR5 antibody and the cancer or precancerous proliferative disorder is breast cancer, triple negative breast cancer, ovarian cancer, or prostate cancer.

9. The method of claim 4, wherein the monoclonal antibody is an anti-HER3 antibody and the cancer or precancerous proliferative disorder is pancreatic cancer, lung cancer, head and neck cancer, breast cancer, gastric cancer, colorectal cancer, esophageal cancer, or ovarian cancer.

10. The method of claim 4, wherein the monoclonal antibody is an anti-HER2 antibody and the cancer or precancerous proliferative disorder comprises HER2-expressing cancer cells.

11. The method of claim 10, wherein the cancer or precancerous proliferative disorder is a breast cancer or an ovarian cancer.

12. The method of claim 4, wherein the monoclonal antibody is an anti-TROP2 antibody and the cancer or precancerous proliferative disorder comprises TROP2-expressing cancer cells.

13. The method of claim 12, wherein the cancer or precancerous proliferative disorder is pancreatic cancer, lung cancer, head and neck cancer, breast cancer, gastric cancer, colorectal cancer, esophageal cancer, or ovarian cancer.

14. The method of claim 1, wherein the one or more radiolabeled cancer targeting agents comprises a radiolabeled PSMA-targeting agent.

15. The method of claim 14, wherein the cancer or precancerous proliferative disorder is a prostate cancer.

16. The method of claim 1, wherein the one or more CD47 blockades are discrete molecules from the one or more therapeutically radiolabeled cancer targeting agents.

17. The method of claim 1, wherein a therapeutically radiolabeled cancer targeting agent f the one or more therapeutically radiolabeled cancer targeting agents targets one or more of CD33, DR5, 5T4, HER2 (ERBB2; Her2/neu), HER3, TROP2, mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), LYPD3 (C4.4A), Nectin-4, urokinase plasminogen activator receptor (uPAR), Folate receptor alpha (FOLR1), CUB-domain containing protein 1 (CDCP1), Glypican-3 (GPC3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H₃, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), MUC1, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECi2A, BST2, EMR2, LY75, GPC3, FCRL5, GPA7, and IGLL1.

18. The method of claim 17, wherein a therapeutically radiolabeled cancer targeting agent is labeled with an alpha particle emitting radionuclide.

19. The method of claim 17, wherein a therapeutically radiolabeled cancer targeting agent is labeled with a beta particle emitting radionuclide.

20. The method of claim 16, wherein the CD47 blockade is a discrete molecule from the therapeutically radiolabeled cancer targeting agent.
